SEQUENCE LISTING (1 sequence)

SEQ ID NO: 1 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

(19) United States
(12) Patent Application Publication
(10) Pub. No.: US 2023/0218572 A1
(43) Pub. Date: Jul. 13, 2023

(54) COMPOSITIONS AND METHODS FOR IMPROVED MESENCHYMAL STEM CELL THERAPY

(71) Applicants: Versitech Limited, Hong Kong (CN); The Chinese University of Hong Kong, Hong Kong (CN)

(72) Inventors: Chun Ming Wu, Hong Kong (CN); Chi Fung Chan, Hong Kong (CN); Ellen Ngar Yun Poon, Hong Kong (CN)

(21) Appl. No.: 18/063,604

(22) Filed: Dec. 8, 2022

Related U.S. Application Data
(60) Provisional application No. 63/287,402, filed on Dec. 8, 2021.

(51) Int. Cl. A61K 31/40; A61K 31/517; A61P 37/06

(52) U.S. Cl. CPC A61K 31/40; A61K 31/517; A61P 37/06

(57) ABSTRACT

It has been established that CD55 expression on human mesenchymal stem cells (hMSCs) is positively correlated with successful allogenic transplantation procedures. Compositions and methods for improved hMSCs, having increased expression of CD55 mRNA and/or increased expression of CD55 on the surface of the cell as compared to a control cell, are provided. Methods include isolating hMSCs from a tissue that naturally has increased CD55 expression, such as adipose tissue derived hMSC (AT-hMSC); and/or in vitro culturing hMSCs in the presence of one or more active agents that stimulates or enhances the expression of CD55 mRNA, and/or increases the surface expression of CD55 in the hMSC to provide improved hMSCs. Preferred active agents include HMG-CoA reductase inhibitors such as atorvastatin and EGFR inhibitors such as erlotinib. Compositions and methods for administering the improved hMSCs to a subject in need thereof are also provided.

### CROSS-REFERENCE TO RELATED APPLICATIONS

This application claims the benefit of and priority to U.S. Ser. No. 63/287,402, filed on Dec. 8, 2021, which is incorporated by reference herein in its entirety.

### REFERENCE TO SEQUENCE LISTING

The Sequence Listing submitted as an xml file named “UHK_01094.xml,” created on Dec. 1, 2022, and having a size of 2,093 bytes is hereby incorporated by reference pursuant to 37 C.F.R. § 1.834(c)(1).

### FIELD OF THE INVENTION

The invention is generally directed to stem cell therapies including improved mesenchymal stem cells.

### BACKGROUND OF THE INVENTION

Graft-versus-Host Disease (GVHD) is a common disease affecting approximately 30 to 40 percent of patients who are transplanted with cells from a related donor, and approximately fifty percent of patients who are transplanted with cells from an unrelated donor. The development of GVHD starts with tissue damage induced by the condition regimen, which then activation host's T cell leading to the migration of T cells to targeted organs. Eventually, organs further deteriorate and ending up with organ failure.

There are two forms of GVHD clinically, acute GVHD and chronic GVHD. Acute GVHD occurs within 100 days after allogenic transplantation and the survival rate of severe acute GVHD patients is approximately 50% on average within 2 years. Although steroid is considered as standard therapy for acute GVHD, it may fail in particularly when patient is steroid-resistant.

Transplantation of human mesenchymal stem cells (hMSCs) can be used to treat a variety of degenerative and immune disorders, including GVHD, but is limited by its limited passage capacity for scale-up clinical application and suboptimal survival in vivo. In 2004, the first report on using hMSCs to treat steroid refractory acute GVHD was released. Bone marrow-derived hMSC (BM-hMSC) from the patient's parent was added and it successfully ameliorated the development of acute GVHD. Since then, hMSC have been used as an adjunct for refractory acute GVHD in some centers.

Currently, bone marrow-derived hMSC are used as the primary source for hMSC. hMSCs are currently used in a large number of clinical trials as therapeutic agents for immune and degenerative diseases, and their efficacy against aGVHD was recently supported in phase III clinical trials. This trial, and most others, utilize BM-hMSCs, but hMSCs can also be derived from umbilical cord (UC) and adipose tissue (AT) and the efficacies of the latter are unclear. Systemic clinical application of hMSCs such as treatment for aGVHD requires 1 to 2 million cells/kg of body weight, representing a large number of cells. Therefore, the proliferation potential of hMSCs is a crucial determinant for clinical application. Proliferation potential can also affect the ability of hMSCs to survive and engraft after transplantation, therefore implicates on treatment outcome. It is important to find the optimal source of hMSCs for aGVHD to increase the clinical efficacy or effectiveness on treating aGVHD using hMSC.

However, the removal of bone marrow is an unpleasant and invasive procedure that limits the amount of BM-hMSC available in the clinic. hMSC isolated from different sources demonstrate similar characteristics in terms of spindle-shaped morphology, specific surface markers expression, and trilineage differentiation into adipocyte, chondrocyte, and osteocytes. However, it is still unclear whether hMSC from different sources can be applied clinically in a similar fashion. A chip-seq study has compared the transcriptional level between BM-hMSC and umbilical cord-derived hMSC (UC-hMSC), found that there was a difference in transcriptomic profile between these two hMSCs. These transcriptional differences may be translated into differences in phenotype leading to variation in clinical effectiveness. Besides, another study also reported that there is a difference in transcriptomic profile difference between adipose tissue-derived hMSC (AT-hMSC) and BM-hMSC, although they match in basic characteristics. In any event, complement activation by the host immune system can induce cellular lysis of transplanted MSCs and therefore reduces the efficacy of MSC therapy. Thus, there remains a critical need to improve the efficacy of MSC therapy and reduce cellular lysis of transplanted MSCs.

Therefore, it is an object of the invention to provide compositions and methods of use thereof for improving the efficacy of MSC therapy.

It is another object to provide compositions and methods for treating and/or preventing one or more of the pathological processes associated with graft versus host disease.

It is a further object to provide methods for selecting and/or identifying candidate cells and tissues with reduced risk of complement-mediated cell lysis for use in allogenic transplantation procedures.

### SUMMARY OF THE INVENTION

It has been established that CD55 expression on human mesenchymal stem cells (hMSCs) is positively correlated with successful allogenic transplantation procedures. CD55 has been demonstrated to be a marker for hMSCs which have high transplantation potential. Therefore, pharmaceutical upregulation of CD55 expression on hMSC is described for improved allogenic transplantation of hMSCs.

Compositions and methods for providing an improved human mesenchymal stem cell (hMSC), having increased expression of CD55 mRNA and/or increased expression of CD55 on the surface of the cell as compared to a control cell are described. The methods include a step of contacting an hMSC with one or more active agents that induce or stimulate expression of CD55 mRNA and/or increase expression of CD55 on the surface of the cell as compared to a control cell. In some forms, the methods also include the step of selecting or purifying the improved hMSC from the culture media and optionally quantitating the level of CD55 expressed in the hMSC. Preferably, the active agent that induces or stimulates expression of CD55 mRNA and/or increases expression of CD55 on the surface of the cell is an EGRF inhibitor or an HMG-CoA reductase inhibitor. Exemplary HMG-CoA reductase inhibitors include atorvastatin, fluvastatin, lovastatin, pravastatin, rosuvastatin, simvastatin, and pitavastatin. In a preferred form, the HMG-CoA reductase inhibitor is atorvastatin. Typically, atorvastatin is present within culture media at an amount between about 2 μM and 50 μM, inclusive, preferably between 5 μM and 25 μM, inclusive. Exemplary EGRF inhibitors include erlotinib, afatinib, brigatinib, icotinib, osimertinib, neratinib, gefitinib, cetuximab, panitumumab, lapatinib, dacomitinib, necitumumab, and vandetanib. In a preferred form, the EGRF inhibitor is erlotinib. Typically, erlotinib is present within culture media at an amount between about 2 μM and 25 μM, inclusive, preferably between 5 μM and 10 μM, inclusive. The compositions of an improved human mesenchymal stem cell (hMSC), having increased expression of CD55 mRNA and/or increased expression of CD55 on the surface of the cell as compared to a control cell, prepared according to the disclosed methods are also described.

Methods of selecting a human mesenchymal stem cell (hMSC) for allogenic transplantation are also provided. Typically, the methods include (a) measuring the level of CD55 mRNA and/or the level of expression of CD55 glycoprotein on the surface of the cell; and (b) selecting the hMSC as one suitable for transplantation if the level of CD55 mRNA and/or the level of expression of CD55 glycoprotein on the surface of the cell is higher than a reference or a control cell. In some forms, the hMSC is isolated from one or more tissues such as bone marrow, adipose tissue, amniotic fluid, endometrium, dental tissues, umbilical cord, Wharton's jelly, amniotic membrane, limb bud, menstrual blood, peripheral blood, placenta and fetal membrane, salivary gland, skin, foreskin, sub-amniotic umbilical cord lining membrane, and synovial fluid. In a preferred form, the hMSC is isolated from adipose tissue. In some forms, the methods also include the step of administering the selected hMSCs to a recipient subject in need thereof, for example, a subject who is undergoing allogenic transplantation or is suffering from one or more symptoms associated with acute graft versus host disease.

Methods of treating, retarding development of, or preventing development of one or more degenerative and immune disorders in a subject have also been developed. The methods include administering to the subject in need thereof an effective amount of human mesenchymal stem cells (hMSCs), having increased expression of CD55 mRNA and/or increased expression of CD55 on the surface of the cell as compared to a control cell. In a preferred form, the hMSCs are isolated from adipose tissue. In other forms, the hMSCs are cultured with one or more active agents that induce or stimulate expression of CD55 mRNA and/or increase expression of CD55 on the surface of the cell as compared to a control cell. In some forms, the one or more active agents are HMG-CoA reductase inhibitors and EGRF inhibitors. Exemplary HMG-CoA reductase inhibitors include atorvastatin, fluvastatin, lovastatin, pravastatin, rosuvastatin, simvastatin, and pitavastatin. In a preferred form, the HMG-CoA reductase inhibitor is atorvastatin. Exemplary EGRF inhibitors include erlotinib, afatinib, brigatinib, icotinib, osimertinib, neratinib, gefitinib, cetuximab, panitumumab, lapatinib, dacomitinib, necitumumab, and vandetanib. In a preferred form, the EGRF inhibitor is erlotinib. In some forms, the one or more degenerative and immune disorders are one or more neurodegenerative diseases and autoimmune diseases. Exemplary neurodegenerative diseases include amyotrophic lateral sclerosis, Parkinson's disease, muscular dystrophy, Alzheimer disease. Exemplary autoimmune diseases include rheumatoid arthritis, Crohn's disease, ulcerative colitis, and Type 1 diabetes. In preferred forms, the degenerative and immune disorder is graft versus host disease (GvHD), such as acute GvHD.

### BRIEF DESCRIPTION OF THE DRAWINGS

FIGS. 1A-1B are graphical representations of differences in gene expression profiles amongst human mesenchymal stem cells (hMSCs) derived from each of Bone Marrow (BM), Umbilical cords (UC), and Adipose Tissue (AT). FIG. 1A shows numbers of differentially expressed genes (DEGs) between in each of AT-hMSC and BM-hMSC (2318), UC-hMSC and BM-hMSC (2270), and AT-hMSC and UC-hMSC (2286), respectively. FIG. 1B is a bar graph showing number of differentially up-regulated and down-regulated genes between each of AT-hMSC and BM-hMSC, UC-hMSC and BM-hMSC, and AT-hMSC and UC-hMSC, respectively.

FIGS. 2A-2B are Venn diagrams showing differentially expressed genes (DEGs) between BM-hMSC and AT-hMSC or UC-hMSC, using expression data from 3 public datasets and the test dataset, with top differentially expressed genes indicated in tables at left. FIG. 2A is a Venn diagram showing the comparison of AT-hMSC vs BM-hMSC. FIG. 2B is a Venn diagram showing the comparison of UC-hMSC vs BM-hMSC. The presence of CD55 amongst both Venn diagrams is highlighted. Up regulated genes are screened out by log fold change >1, P<0.05.

FIGS. 3A-3B are bar graphs showing CD55 mRNA and surface protein expression of hMSCs. FIG. 3A is a bar graph of CD55 mRNA expression of hMSCs normalized to GADPH (0-3) for each MSC group (Adipose Tissue; AT, Bone Marrow; BM, and Umbilical Cord; UC), respectively. Graph shows data (mean+/−SEM) of three experiments performed in triplicate. FIG. 3B is a bar graph of CD55 surface protein expression, showing % CD55+ cells (0-100) for each of Adipose Tissue (AT), Bone Marrow (BM), and Umbilical Cord (UC)-derived MSCs, respectively. Graphs show data of three experiments with mean+/−SEM. * p<0.05, ** p<0.01, *** p<0.001, **** p<0.0001.

FIGS. 4A-4C are histograms of flow cytometry analysis showing cell counts for CD59 surface protein expression on hMSCs derived from each of Adipose Tissue (AT; FIG. 4A), Bone Marrow (BM; FIG. 4B), and Umbilical Cord (UC; FIG. 4C), respectively.

FIGS. 5A-5B are bar graphs showing C3 deposition on hMSCs upon mouse serum treatment. FIG. 5A is a bar graph of C3 deposition of hMSC on cell surface, showing % of C3b expression (0-30) for each MSC group (Adipose Tissue; AT, Bone Marrow; BM, and Umbilical Cord; UC), respectively. FIG. 5B is a bar graph of cytotoxicity assay of the serum treatment on hMSC accessed by BCECF, AM, showing % cytotoxicity (0-30) for each of Adipose Tissue (AT), Bone Marrow (BM), and Umbilical Cord (UC)-derived MSCs, respectively. Graphs show data of three experiments with mean+/−SEM. * p<0.05, ** p<0.01, *** p<0.001.

FIGS. 6A-6I are histograms of flow cytometry analysis showing cytometry analysis of hMSC derived from different tissues with or without CD55 antibody with mouse serum treatment. FIG. 6A is the unstained control; FIG. 6B is hMSC treated with mouse serum without CD55 antibody blocking, and FIG. 6C is hMSC treated with mouse serum with CD55 antibody blocking, of hMSCs derived from Adipose Tissue. FIG. 6D is the unstained control; FIG. 6E is hMSC treated with mouse serum without CD55 antibody blocking and FIG. 6F is hMSC treated with mouse serum with CD55 antibody blocking, of hMSCs derived from Bone Marrow. FIG. 6G is the unstained control; FIG. 6H is hMSC treated with mouse serum without CD55 antibody blocking and FIG. 6I is hMSC treated with mouse serum with CD55 antibody blocking, of hMSCs derived from Umbilical Cord.

FIG. 7 is a bar graph of C3 deposition of hMSC on cell surface with CD55 antibody blocking, showing % of C3b expression (0-100) for hMSCs derived from each of Adipose Tissue, Bone Marrow, and Umbilical Cord, respectively.

FIG. 8 is a linear regression plot of % of C3 deposition and % of CD55 positive cells in hMSCs derived from each of Adipose Tissue (AT), Bone Marrow (BM), and Umbilical Cord (UC), respectively, as well as all hMSCs combined.

FIG. 9 is a bar graph of CD55 mRNA expression of hMSCs in presence of atorvastatin, showing % Normalized to GADPH (0-25) for each MSC group Adipose Tissue (AT; red), Bone Marrow (BM; blue), and Umbilical Cord (UC; green), respectively, in the presence of 0 μM (untreated control) 2 μM, 5 μM, 25 μM, and 50 μM atorvastatin. Graph shows data (mean+/−SEM) of three to four experiments performed in triplicate. * p<0.05, ** p<0.01, *** p<0.001, **** p<0.0001.

FIG. 10 is a bar graph of CD55 mRNA expression of hMSCs in presence of Erlotinib, showing % Normalized to GADPH (0-25) for each MSC group Adipose Tissue (AT; red), Bone Marrow (BM; blue), and Umbilical Cord (UC; green), respectively, in the presence of 0 μM (untreated control) 2 μM, 5 μM, and 10 μM Erlotinib. Graph shows data (mean+/−SEM) of three to four experiments performed in triplicate. * p<0.05, ** p<0.01, *** p<0.001, **** p<0.0001.

FIG. 11 is a bar graph of CD55 surface expression on hMSCs following 24 hr in presence of Atorvastatin (Atr) or Erlotinib (Erl), showing % of CD55+ cells (0-100) for each MSC group Adipose Tissue (AT; red), Bone Marrow (BM; blue), and Umbilical Cord (UC; green), respectively, in the presence of nothing (untreated control) 5 μM Atr, or 5 μM Erl. Graph shows data (mean+/−SEM) of five experiments. * p<0.05, ** p<0.01, *** p<0.001, **** p<0.0001.

FIGS. 12A-12B are diagrams depicting preparation of a mouse model of Graft versus host disease (GVHD). FIG. 12A is timeline depicting the order of protocols from D-7 (7 days prior to transplantation) through sacrifice for tissue harvesting at D+80 (80 days following transplantation). Three doses of injection of hMSCs at day 0, 3, 6 for day 21 and 80 timepoint, whereas 1 dose of injection for day 7 timepoint. FIG. 12B is a cartoon diagram depicting the flow of events required for preparation of a mouse model of Graft versus host disease (GVHD) in each of three groups; T-cell depleted bone marrow (TCDBM) only; TCDBM plus Donor CD4+ T cells; and TCDBM plus Donor CD4+ T cells plus mesenchymal stem cells (MSC) from either Adipose Tissue (AT), Bone Marrow (BM), or Umbilical Cord (UC), respectively.

FIG. 13 is a line graph of mouse survival (0-100%) over Days (0-80) following transplantation for each group, including TCDBM (T-cell depleted bone marrow only); TCDBM+CD4 (T-cell depleted bone marrow only plus Donor CD4 T cells); and TCDBM+CD4 plus Donor CD4+ T cells plus mesenchymal stem cells (MSC) from either Adipose Tissue (TCDBM+CD4+AT-MSC), Bone Marrow (TCDBM+CD4+BM− MSC), or Umbilical Cord (TCDBM+CD4+UC-MSC), respectively. n=12. * p<0.05, ** p<0.01.

FIG. 14 is a line graph of body weight (% BW loss) over time (0-80 Days) for each of 5 cohorts, including TCDBM (T-cell depleted bone marrow only; •); TCDBM+CD4 (T-cell depleted bone marrow only plus Donor CD4 T cells; (▪); and TCDBM+CD4 plus Donor CD4+ T cells plus mesenchymal stem cells (MSC) from either Adipose Tissue (TCDBM+CD4+AT-MSC; ▴), Bone Marrow (TCDBM+CD4+ BM− MSC; ▾), or Umbilical Cord (TCDBM+CD4+UC-MSC; ♦), respectively. n=12. * p<0.05, ** p<0.01.

FIG. 15 is a line graph of GVHD score (as described in Table 1) over time (0-80 Days) for each of 5 cohorts, including TCDBM (T-cell depleted bone marrow only; •); TCDBM+CD4 (T-cell depleted bone marrow only plus Donor CD4 T cells; (▪); and TCDBM+CD4 plus Donor CD4+ T cells plus mesenchymal stem cells (MSC) from either Adipose Tissue (TCDBM+CD4+AT-MSC; ▴), Bone Marrow (TCDBM+CD4+ BM− MSC; ▾), or Umbilical Cord (TCDBM+CD4+UC-MSC; ♦), respectively.

### DETAILED DESCRIPTION OF THE INVENTION

### I. Definitions

The term “combination therapy” refers to treatment of a disease or symptom thereof, or a method for achieving a desired physiological change, including administering to an animal, such as a mammal, especially a human being, an effective amount of two or more chemical agents or components to treat the disease or symptom thereof, or to produce the physiological change, wherein the chemical agents or components are administered together, such as part of the same composition, or administered separately and independently at the same time or at different times (i.e., administration of each agent or component is separated by a finite period of time from each other).

The term “dosage regime” refers to drug administration regarding formulation, route of administration, drug dose, dosing interval and treatment duration.

The terms “individual”, “host”, “subject”, and “patient” are used interchangeably, and refer to a mammal, including, but not limited to, murines, simians, humans, mammalian farm animals, mammalian sport animals, and mammalian pets.

The term “effective amount” or “therapeutically effective amount” refers to the amount which is able to treat one or more symptoms of a disease or disorder, reverse the progression of one or more symptoms of a disease or disorder, halt the progression of one or more symptoms of a disease or disorder, or prevent the occurrence of one or more symptoms of a disease or disorder in a subject to whom the formulation is administered, for example, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; etc.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including therapeutic agents may inhibit or reduce one or more markers of a disease or disorder in a subject by about 10%, 20%, 30%, 40%, 50%, 75%, 85%, 90%, 95%, or 99% from the activity and/or quantity of the same marker in subjects that did not receive, or were not treated with the compositions. In some forms, the inhibition and reduction are compared according to the level of mRNAs, proteins, cells, tissues and organs.

The terms “treating” or “retarding development of” in the context of a disease or disorder mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated with HCC are mitigated or eliminated, including, but are not limited to, reducing and/or inhibiting rate of tumor cell proliferation/growth, increasing the quality of life of those suffering from the disease, decreasing the dose of other medications required to treat the disease, delaying the progression of the disease, and/or prolonging survival of individuals.

The term “expressed” or “expression” refers to the transcription from DNA to an RNA nucleic acid molecule at least complementary in part to a region of one of the two nucleic acid strands of the gene. The term “expressed” or “expression” also refers to the translation from said RNA nucleic acid molecule to give a protein or polypeptide or a portion thereof.

The terms “subject,” “individual,” and “patient” refer to any individual who is the target of treatment using the disclosed compositions and methods. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### II. Compositions

Compositions of improved human mesenchymal stem cells (hMSC) for use in allogenic transplantation have been developed. The improved hMSC include enhanced expression of CD55 mRNA and/or enhanced surface presentation of CD55 proteins relative to control hMSC. Compositions of active agents for enhancing CD55 in hMSC are also provided. Typically, the compositions include one or more drugs that up-regulate CD55 in hMSC in vitro and/or in vivo. In some forms, the active agents include one or more EGFR inhibitors. In other forms, the active agents include one or more statins. In further forms, the active agents include HMG-CoA reductase inhibitors. Preferred active agents include erlotinib and atorvastatin.

A. Improved Mesenchymal Stem Cells

Improved human mesenchymal stem cells (hMSCs) are described. The improved hMSCs have greater expression of CD55 and/or reduced surface deposition of complement c3 as compared to control hMSCs.

1. Human Mesenchymal Stem Cells (hMSC)

HMSCs are non-hematopoietic, multipotent stem cells with the capacity to differentiate into mesodermal lineage such as osteocytes, adipocytes, and chondrocytes as well ectodermal (neurocytes) and endodermal lineages (hepatocytes). The International Society for Cellular Therapy (ISCT) has proposed minimum criteria to define MSCs, including (a) should exhibits plastic adherence (b) possess cell surface markers, including cluster of differentiation (CD)73, CD90, and CD105; (c) lack expression of CD14, CD34, CD45, and human leucocyte antigen-DR (HLA-DR); and (d) have the ability to differentiate in vitro into adipocyte, chondrocyte, and osteoblast. These characteristics are valid for all MSCs, although a few differences exist in MSCs isolated from various tissue origins.

a. Sources of MSCs

Typically, hMSCs are isolated from a human donor subject and cultured in vitro prior to being selected and/or “improved”. In some forms, hMSCs are isolated from a donor subject, with a view to being transplanted into a recipient subject. In some forms, the hMSCs are isolated from a donor subject who is related to the potential recipient subject. In other forms, the hMSCs are isolated from a donor subject who has one or more biological/genetic characteristics that is suited or matched to one or more biological/genetic characteristics of the potential recipient subject. For example, in some forms, the donor subject has the same or compatible blood type and/or the same or compatible major histocompatibility type as the potential recipient subject. In other forms, the donor subject is unknown or has an undefined genotype and/or phenotype, or the potential recipient subject is unknown or has an undefined genotype and/or phenotype, or both the donor and potential recipient subject are unknown or have an undefined genotype and/or phenotype. Typically, the donor and potential recipient subjects are different (i.e., allogenic transplantation of hMSCs). However, in some forms, the recipient and donor subjects are the same individual (i.e., autologous transplantation of hMSCs). For example, in some forms, the hMSCs are isolated from an individual at a time or from a tissue that is distinct from the time or tissue that is subject of transplantation. In a particular form, hMSCs are obtained from an individual in the form of stem cells, for example, from umbilical cord, or bone marrow, or adipose tissue, for use in the same individual following one or more procedures to “improve” the hMSCs. In an exemplary form, hMSCs are isolated and frozen/stored for a period of time prior to being re-introduced back into the same individual.

The hMSCs are typically isolated from a specific tissue or organ type. However, in some forms, the hMSCs include stem cells from more than a single tissue. In some forms, hMSCs are isolated from one or more tissues including bone marrow (BM), adipose tissue (AT), amniotic fluid (AF), endometrium (EM), dental tissues (DT), umbilical cord (UC) and Wharton's jelly (WJ), amniotic membrane (AM), limb bud (LB), menstrual blood (MB), peripheral blood (PB), placenta and fetal membrane (PM/FM), salivary gland (SG), skin (S) and foreskin (FS), sub-amniotic umbilical cord lining membrane, and synovial fluid (SF) (Ullah, et al., Bioscience reports, vol. 35,2 e00191. 28 Apr. 2015, doi:10.1042/B5R20150025).

The hMSCs are viable in vitro and are typically between passage number 1 and passage 50, for example, between passage 5 and passage 30, or between passage 10 and 20. Preferably, the hMSCs are not older than 30 days, for example, passaged for between 1 and 21 days in vitro, between 1 and 14 days in vitro or between 1 and 7 days in vitro, inclusive.

Typically, the hMSCs are cultured in vitro. Suitable reagents and methods for culturing hMSCs in vitro are well known in the art (see, for example, Ullah, et al., Bioscience reports, vol. 35,2 e00191. 28 Apr. 2015, doi:10.1042/BSR20150025, the contents of which are incorporated by reference herein).

In some forms, the hMSCs are frozen, for example, in the presence of cryoprotectant. Cryoprotective agents (CPAs) required to prevent any freezing damage to cells are well known in the art (see, for example, Fuller, Cryo. Lett. 2004; 25:375-388, the contents of which are incorporated by reference herein). DMSO is the most common CPAs used in cryopreservation of MSCs. Therefore, in some forms, hMSCs are frozen in one or more cryo-preservatives including DMSO.

b. Immunomodulatory Capabilities of MSCs

Typically, MSCs have immunomodulatory features, secrete cytokines and immune-receptors which regulate the microenvironment in the host tissue, have multilineage potential, immunomodulation and secretion of anti-inflammatory molecules. hMSCs have the capacity to differentiate into all the three lineages, i.e., ectoderm, mesoderm, and endoderm, with various potency by employing suitable media and growth supplements which initiate lineage differentiation.

Due to low expression of MHC class I and lack expression of MHC class II along with co-stimulatory molecules, like CD80, CD40 and CD86, MSCs are unable to bring substantial alloreactivity and these features protect MSCs from natural killer (NK) cells lysis (Rasmusson, et al., Transplantation. 2003; 76:1208-1213. doi: 10.1097/01.TP.0000082540.43730.80). MSCs therapy might alleviate disease response by increasing the conversion from Th2 (T helper cells) response to Th1 cellular immune response through modulation of interleukin (IL)-4 and interferon (IFN)-γ levels in effector T-cells. MSCs have the ability to inhibit the NK cells and cytotoxic T-cells by means of different pathways. The secretion of human leucocytes antigen G5 was also found helpful in the suppression of T lymphocytes and NK cells. By the secretion of suppressors of T-cells development, inhibitory factors, i.e., leukemia inhibitory factor (LIF) and IFN-γ enhance immunomodulatory properties of MSCs. Moreover, human BM-MSCs are not recognized by NK cells, as they express HLA-DR molecules. Therefore, when allogenic hMSCs are transplanted into patients, there is typically no production of anti-allogeneic antibody, nor T-cell priming. However, cytotoxic immune factors are found to be involved in the lysis of MSCs. In this situation, IFN-γ acts as antagonist of NK cells, i.e., IL-2-treated NKs are recognized to destroy MSCs whereas IFN-γ helps the MSCs to keep it safe from NKs. Together with the protection of MSCs from cytotoxic factors, IFN-γ also enhances the differentiation of these cells by the nuclear factor kappa β (NFκB)-dependent and -independent pathways. Toll-like receptors (TLRs) are the key components of the innate immune system, which is critically involved in the initiation of adaptive immune system responses. MSCs express TLRs that elevate their cytokines secretions as well as proliferation. MHC class I chain-like gene A (MICA) together with TLR3 ligand and other immunoregulatory proteins kept the MSCs safe from NKs invasion. Together with other properties, these immunomodulatory features make MSCs one of the feasible stem cell sources for performing cell transplantation experiments.

2. Complement Decay Accelerating Factor (DAF)/CD55

Improved human mesenchymal stem cells (hMSCs) having an increased expression of CD55, or an increased surface expression of CD55 as compared to control cells are described.

CD55, also known as DAF, is a 65-75 kDa glycoprotein that is expressed at the surface of all cells in contact with serum. The amino acid sequence for the mature human CD55 glycoprotein is set forth below:

```
(SEQ ID NO: 1)
              10 20 30 40
MTVARPSVPA ALPLLGELPR LLLLVLLCLP AVWGDCGLPP
              50 60 70 80
DVPNAQPALE GRTSFPEDTV ITYKCEESFV KIPGEKDSVI
              90 100 110 120
CLKGSQWSDI EEFCNRSCEV PTRLNSASLK QPYITQNYFP
                130 140 150 160
VGTVVEYECR PGYRREPSLS PKLTCLQNLK WSTAVEFCKK
                170 180 190 200
KSCPNPGEIR NGQIDVPGGI LFGATISFSC NTGYKLFGST
                210 220 230 240
SSFCLISGSS VQWSDPLPEC REIYCPAPPQ IDNGIIQGER
                250 260 270 280
DHYGYRQSVT YACNKGFTMI GEHSIYCTVN NDEGEWSGPP
                290 300 310 320
PECRGKSLTS KVPPTVQKPT TVNVPTTEVS PTSQKTTTKT
                330 340 350 360
TTPNAQATRS TPVSRTTKHF HETTPNKGSG TTSGTTRLLS
                370 380
GHTCFTLTGL LGTLVTMGLL T.
```

a. Function of CD55 in hMSC

hMSC with increased expression and/or surface presentation of CD55 exhibit enhanced CD55 function. CD55 recognizes complement (C4b and C3b) fragments that condense with cell-surface hydroxyl or amino groups when nascent C4b and C3b are locally generated during C4 and C3 activation. Interaction of DAF with cell-associated C4b and C3b polypeptides interferes with their ability to catalyze the conversion of complement C2 and factor B to enzymatically active C2a and Bb and thereby prevents the formation of C4b2a and C3bBb, the amplification convertases of the complement cascade.

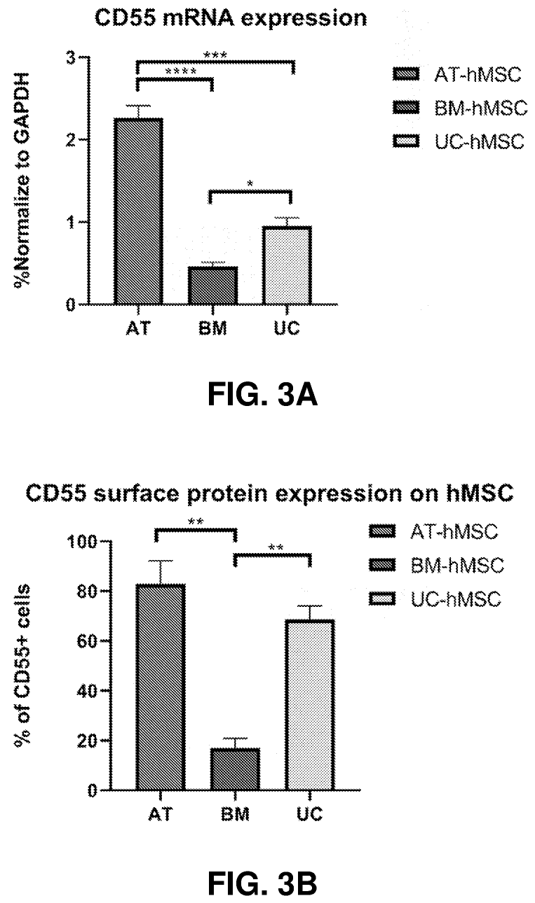

As described in the Examples, experimental data established that all of Adipose Tissue derived hMSC (AT-hMSC), Bone Marrow derived hMSC (BM-hMSC), and Umbilical cord derived hMSC (UC-hMSC) expressed CD55 surface proteins. Among all hMSC, Adipose Tissue derived hMSC (AT-hMSC) is significantly higher than Bone Marrow derived hMSC (BM-hMSC) and Umbilical cord derived hMSC (UC-hMSC) in mRNA and surface protein expression (see FIGS. 3A-3B).

b. Increased Expression of CD55

Compositions of hMSCs having increased surface expression of CD55 that exhibit enhanced survival and reduce graft versus host disease following allogenic transplant in vivo are described.

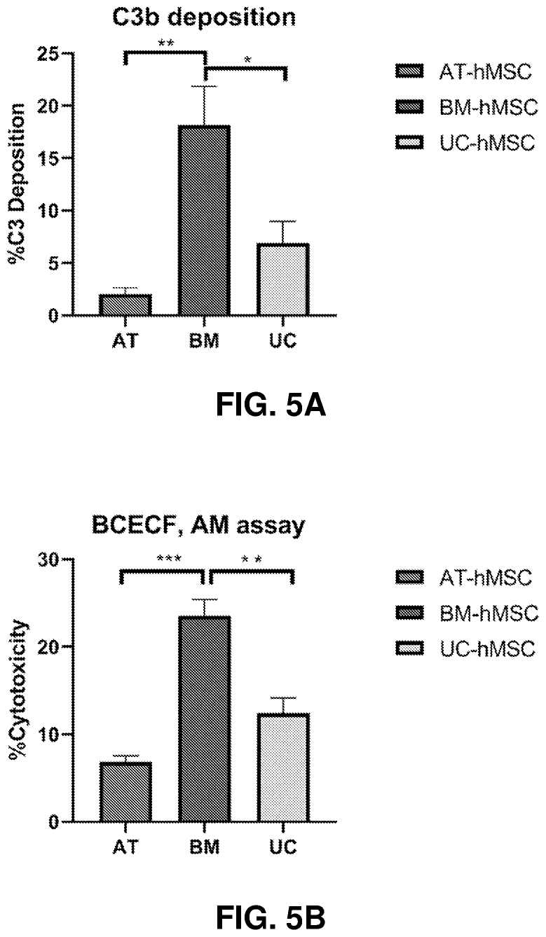

Since CD55 functions to provide a protective barrier threshold against complement activation and C3b deposition on the plasma membranes of “normal” hMSC cells, increasing the expression of CD55 reduces cytotoxicity of hMSC in serum (see FIGS. 5A-5B), and increases hMSC survival following transplantation in vivo. hMSC with higher expression of CD55 have lower C3b deposition than those with lower expression of CD55. Therefore, in some forms, improved hMSCs have increased expression of CD55 at the cell surface relative to control hMSCs. Methods for measuring the relative amount and/or surface density of a cell surface marker on a given cell type are well known in the art. An exemplary method is flow cytometry, for example, as described in the Examples.

In some forms, improved hMSC have an increased intracellular expression of CD55 mRNA, or increased surface presentation of CD55 glycoprotein, or both, as compared to a non-improved hMSC. The amount of surface expression of CD55 can be an average for a single cell, or for a population of cells having a single or multiple cell types. The increase in expression can be a natural increase or can be induced by one or more exogenous compounds. For example, in some forms, an improved hMSC is a hMSC that has increased expression of CD55 mRNA or increased surface expression of CD55 relative to another hMSC cell or type, as the result of a genetic anomaly. In other forms, one type of hMSC derived from a specific lineage or source has improved CD55 expression over another type of hMSC from a different specific lineage or source. For example, as set forth in the Examples, it has been established that AT-hMSCs exhibit increased CD55 expression relative to BM-hMSCs. In other forms, an improved hMSC is a hMSC that has increased expression of CD55 mRNA or increased surface expression of CD55 following exposure to one or more active agents or cells that induces or stimulates an increase in the cellular expression of CD55, or an increase in the function of CD55, or an increase in the amount of CD55 at the cell surface, or combinations thereof. In some forms, a control or non-improved hMSC is an hMSC of the same or different type that does not have increased expression of CD55 mRNA or increased surface expression of CD55. The increase can be from about 1% to about 100%, inclusive, of the amount of CD55 on a control cell or population of control cells. For example, in some forms, an increase is at least 1%, 2%, 3%, 4%, 5%, 10%, 15%, 20%, 30%, 40%, 50%, 60%, 70%, 80%, 90%, at least 100%, or more than 100% of the amount of CD55 on a control cell, such as 200%, 300%, 500% up to 1,000%. In a preferred form, the amount of increase is between 10% and 30%, inclusive, such as 20% of the amount of CD55 at the surface of a control cell, such as the same cell type in the absence of an active agent that increases expression of CD55. The increase in CD55 can be long-lasting following exposure of the cell(s) to the active agent. For example, in some forms, the increase in CD55 expression is observed up to 2 days, 3 days, 5 days, 7 days, 10 days, 14 days, 21 days, 28 days, 30 days, or more than 30 days following exposure to an active agent or cell that increases CD55 expression.

B. Active Agents

Active agents that increase the expression of CD55 mRNA, and/or the surface presentation of CD55 on hMSCs are described. Exemplary active agents include HMG-CoA reductase inhibitors and EGFR inhibitors.

1. HMG-CoA Reductase Inhibitors/Statins

In some forms, the active agent that enhances expression of CD55 on hMSCs in vitro is an HMG-CoA reductase inhibitor, or “statin” drug. Statins, also known as HMG-CoA reductase inhibitors, are a class of lipid-lowering medications that reduce illness and mortality in those who are at high risk of cardiovascular disease. They are the most common cholesterol-lowering drugs, having the common formula set forth in Formula I.

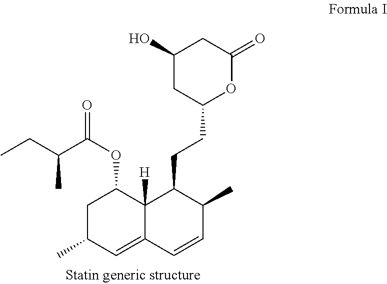 [SMILES: CC[C@H](C)C(=O)O[C@H]1C[C@@H](C)C=C2C=C[C@H](C)[C@H](CC[C@@H]3C[C@@H](O)CC(=O)O3)[C@H]21]

Exemplary statins include Atorvastatin (LIPITOR®), Fluvastatin (LESCOL®, LESCOL XL®), Lovastatin (MEVACOR®, ALTOPREV®), Pravastatin (PRAVACHOL®), Rosuvastatin (CRESTOR®), Simvastatin (ZOCOR®), or Pitavastatin (LIVALO®). A preferred statin/HMG-CoA reductase inhibitors is atorvastatin.

a. Atorvastatin

In some forms, the active agent that enhances expression of CD55 on hMSCs in vitro is atorvastatin. It has been established that treatment with the drug atorvastatin on hMSC induces a significantly higher expression of CD55 than untreated control.

Atorvastatin is a statin used to prevent cardiovascular disease in patients having high risk of cardiovascular disease. It is commercially available as an oral formulation sold under the name LIPITOR®. Atorvastatin (CAS number 134523-00-5) is a completely synthetic compound having a molar mass of 558.64 g, chemical formula C33H35FN205, and a structure as set forth in Formula II.

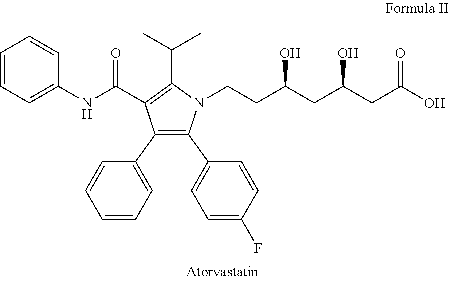 [SMILES: CC(C)c1c(C(=O)Nc2ccccc2)c(-c2ccccc2)c(-c2ccc(F)cc2)n1CC[C@@H](O)C[C@@H](O)CC(=O)O]

Atorvastatin is a competitive inhibitor of HMG-CoA reductase HMG-CoA reductase catalyzes the reduction of 3-hydroxy-3-methylglutaryl-coenzyme A (HMG-CoA) to mevalonate, which is the rate-limiting step in hepatic cholesterol biosynthesis. Inhibition of the enzyme decreases de novo cholesterol synthesis, increasing expression of low-density lipoprotein receptors (LDL receptors) on hepatocytes. This increases LDL uptake by the hepatocytes, decreasing the amount of LDL-cholesterol in the blood. Like other statins, atorvastatin also reduces blood levels of triglycerides and slightly increases levels of HDL-cholesterol.

In some forms, atorvastatin is present within culture media at between 1 μM and 100 μM, for example, between about 2 μM and 50 μM, inclusive, for example, between 5 μM and 25 μM, inclusive.

2. Epidermal Growth Factor Receptor (EGFR) Inhibitors

In some forms, the active agent that enhances expression of CD55 on hMSCs in vitro is an epidermal growth factor receptor (EGFR) inhibitor. The epidermal growth factor receptor (EGFR; ErbB-1; HER1 in humans) is a transmembrane protein that is a receptor for members of the epidermal growth factor family (EGF family) of extracellular protein ligands. The epidermal growth factor receptor is a member of the ErbB family of receptors, a subfamily of four closely related receptor tyrosine kinases.

Epidermal growth factor receptor (EGFR) is a transmembrane protein that is activated by binding of its specific ligands, including epidermal growth factor and transforming growth factor α (TGFα). ErbB2 has no known direct activating ligand and may be in an activated state constitutively or become active upon heterodimerization with other family members such as EGFR. Upon activation by its growth factor ligands, EGFR undergoes a transition from an inactive monomeric form to an active homodimer. Multiple EGFR inhibitors are commercially available, including erlotinib, afatinib, brigatinib, icotinib, osimertinib, neratinib, gefitinib, cetuximab, panitumumab, lapatinib, dacomitinib, necitumumab and vandetanib. A preferred EGFR inhibitor is erlotinib.

a. Erlotinib

In some forms, the active agent that enhances expression of CD55 on hMSCs in vitro is erlotinib. It has been established that treatment with the drug erlotinib on hMSC induces a significantly higher expression of CD55 than untreated control.

Erlotinib (CAS number 183321-74-6) has a mass of 393.4 g, a chemical formula C₂₂H₂₃N₃O₄ and a structure as set forth in Formula III.

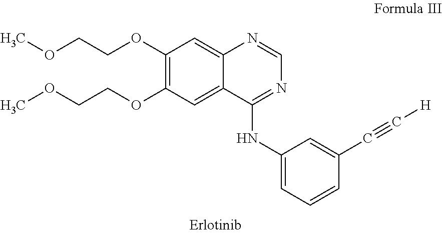 [SMILES: C#Cc1cccc(Nc2ncnc3cc(OCCOC)c(OCCOC)cc23)c1]

In some forms, erlotinib is present within culture media at between 1 μM and 100 μM, inclusive, for example, between about 2 μM and 20 μM, inclusive, for example, between 5 μM and 10 μM, inclusive.

C. Formulations of Improved hMSCs

Formulations of, and pharmaceutical compositions including one or more improved hMSCs having enhanced expression of CD55 are provided. The formulations typically include an amount of improved hMSC, i.e., hMSCs having enhanced expression of CD55 relative to a control hMSC for administration to a subject in need thereof. In some forms, the improved hMSCs are formulated for administration to a subject in vivo together with one or more buffers, preservatives, cell nutrients, culture media, or additional active agents, such as therapeutic, diagnostic, or prophylactic agents. Typically, the formulations of improved hMSC are pharmaceutical compositions. Therefore, in some forms, the pharmaceutical compositions of improved hMSCs include one or more additional active agents. For example, in some forms, the pharmaceutical composition of improved hMSCs includes two, three, or more active agents. The pharmaceutical compositions can be formulated as a pharmaceutical dosage unit, referred to as a unit dosage form.

Formulations of combination therapies typically include an effective amount of improved hMSCs for allogenic transplantation into a subject in need thereof. Effective amounts of improved hMSCs and optionally additional active agents are discussed in more detail below. It will be appreciated that in some forms the effective amount of improved hMSCs is different from the amount of control or non-improved hMSCs that would be effective for the same therapeutic or prophylactic efficacy when administered to a recipient subject. For example, in some forms the effective amount of improved hMSCs is a lower dosage than the dosage of control or non-improved hMSCs that is effective when administered to achieve therapeutic or prophylactic efficacy in a recipient subject. In other forms, the control or non-improved hMSCs are not effective to achieve therapeutic or prophylactic efficacy in a recipient subject, and therapeutic or prophylactic efficacy is only achieved with administration of improved hMSCs to the subject.

1. Delivery Vehicles and Devices

The improved hMSCs can be administered and taken up into a tissue or organ of a subject with or without the aid of a delivery vehicle. Appropriate delivery vehicles for hMSCs are known in the art and can be selected to suit the particular needs of the recipient subject. For example, in some forms, the improved hMSCs are incorporated into or encapsulated by a medical device or prosthesis, such as a tissue graft or scaffold. For example, the compositions of improved hMSCs can be incorporated into/onto a medical device which provides controlled release of one or more active agent(s). In some forms, release of the active agents(s) is controlled by diffusion of the active agent(s) out of the device and/or degradation of the device, for example by hydrolysis and/or enzymatic degradation. Suitable polymers for use in such devices include ethylcellulose and other natural or synthetic cellulose derivatives.

Polymers which are slowly soluble and form a gel in an aqueous environment, such as hydroxypropyl methylcellulose or polyethylene oxide may also be suitable as materials for hMSC and/or drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some forms, both improved hMSCs and one or more active agents are incorporated into particles and are formulated for administration in vivo.

2. Pharmaceutical Compositions

Pharmaceutical compositions including improved hMSCs and optionally one or more additional active agent(s) with or without a delivery vehicle are provided. Pharmaceutical compositions can be for administration by parenteral (intramuscular, intraperitoneal, intravenous (IV) or subcutaneous injection) routes of administration or using bioerodible inserts and can be formulated in dosage forms appropriate for administration.

In certain forms, the compositions of improved hMSCs are administered locally, for example, by injection directly into a site to be treated (e.g., into a site of a transplant).

In some forms, the compositions of improved hMSCs are injected or otherwise administered directly into the vasculature onto vascular tissue at or adjacent to the intended site of treatment (e.g., adjacent to a site of a transplant). Typically, local administration causes an increased localized concentration of the improved hMSCs and optionally additional active agents which is greater than that which can be achieved by systemic administration. Targeting of the molecules or formulation can be used to achieve more selective delivery.

a. Formulations for Parenteral Administration

In some forms, improved hMSCs and optionally one or more additional active agent(s) and pharmaceutical compositions thereof are administered in an aqueous solution, by parenteral injection. Formulations of improved hMSCs may be in the form of a suspension or emulsion. In general, pharmaceutical compositions are provided including effective amounts of the improved hMSCs and optionally include pharmaceutically acceptable diluents, preservatives, solubilizers, emulsifiers, adjuvants and/or carriers that support hMSC viability. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized prior to the addition of improved hMSCs by, for example, filtration through a bacteria retaining filter, by incorporating sterilizing agents into the compositions, by irradiating the compositions, or by heating the compositions and cooling prior to addition of improved hMSCs. In some forms, the formulations include one or more reagents used to culture or freeze/thaw the improved hMSCs, such as DMSO.

D. Adjunct and Additional Therapies and Procedures

The compositions of improved hMSCs can be administered to a subject in combination with one or more adjunct therapies or procedures or can be an adjunct therapy to one or more primary therapies or producers. The additional therapy or procedure can be simultaneous or sequential with the administration of improved hMSCs. In some forms, the additional therapy or procedure is surgery, transplant surgery, a radiation therapy, or chemotherapy.

### III. Methods of Use

It has been established that compositions of improved hMSCs having increased surface expression of CD55 exhibit enhanced survival and reduce graft versus host disease following allogenic transplant in vivo.

Methods exploiting the homing ability, multilineage potential, secretion of anti-inflammatory molecules, and immunoregulatory effects of improved MSCs for use in the treatment of autoimmune, inflammatory, and degenerative diseases are described. The methods reduce graft versus host disease associated with the hMSC-based treatment and prevention of autoimmune, inflammatory, and degenerative diseases, by administering to a subject in need thereof an effective amount of improved hMSCs to treat or prevent the one or more autoimmune, inflammatory, and degenerative diseases in the subject. Methods for generating, isolating, and selecting improved hMSCs having enhanced expression of CD55 are also provided. In some forms, the methods expose hMSCs to one or more active agents that increase or enhance the expression of CD55 mRNA and/or give rise to enhanced surface expression of CD55 proteins on the hMSC. In other forms, the methods screen hMSCs in vitro for the amount of surface expression of CD55 and select or isolate the cells having the greatest surface expression of CD55 prior to administering the cells for allogenic transplantation in vivo. In some forms, the step of selection includes removing hMSCs that have the least expression of CD55, for example, removing the lowest 1% to 90% CD 55 expressing cells of the total number of cells, for example, selecting only the highest CD 55 expressing cells as a percentage of the total number of cells, e.g., 99%-1%, such as the highest 60%, the highest 50%, the highest 40%, the highest 30%, the highest 20%, or the highest 10% CD 55 expressing cells of the hMSCs.

A. Methods For Selecting hMSCs for Transplantation

Methods for characterizing hMSCs to determine the amount of or rate of expression of CD55 in hMSCs are provided. Therefore, methods for assessing the potential of an hMSC or a population of hMSCs for successful allogenic or autologous transplantation are also disclosed. The methods for assess a hMSC or population of hMSCs as being potentially more successful when used in allogenic or autologous transplantation if the hMSC have a higher amount of CD55 at the cell surface than a reference or control amount, for example, in a similar hMSC or population of hMSCs that are known to have little or no expression of CD55.

In particular forms, the methods characterize hMSCs in vitro so as to assess the extent to which the hMSCs express CD55. For example, hMSCs that have more or less than a threshold value of CD55 expression can be selected for use in allogenic transplant into a subject in need thereof. In preferred forms, minimum threshold is at least 10% positive expression of CD55 of the MSCs. The survival curve results of the in vivo experiment indicated that all hMSCs treatment have an improvement in survival rate compared with the positive control group, including BM-hMSC even though BM-hMSCs has the lowest expression of CD55 among all sources of hMSCs. Thus, in preferred forms, minimum threshold is at least 10%, 20%, 30%, 40%, 50%, 60%, 70%, 80%, or 90% expression of CD55 on BM-hMSCs.

In some forms, the methods employ one or more well-known analytical techniques to detect and quantitate the expression or presence of CD55 of one or more hMSC in a population of hMSC. Exemplary techniques to quantitate expression include quantitative polymerase chain reaction (q-PCR), for example, as implemented in a real-time q-PCR protocol. Exemplary methods for detecting and quantitating CD55 surface expression include staining with molecular probes and/or imaging, densitometry, flow cytometry, etc.

Methods for comparing different populations of hMSCs for amenability of allogenic transplant into a subject for therapy are also provided. In some forms, the methods include characterizing different populations of hMSCs by determining whether the hMSCs have a greater or lower amount of surface CD55 than a control or threshold value, and selecting and/or isolating the cells or population of cells with the highest levels of CD55 expression for administration to a subject in need thereof.

All of the methods can include one or more steps of identifying a donor subject, from whom cells are obtained, and/or identifying a potential recipient subject, into whom the hMSCs are to be administered. In some forms, subjects having hMSCs that express high levels of CD55 that are greater than an average or threshold value are identified as preferred donors. In some forms, tissues or organs that give rise to hMSCs that express high levels of CD55 that are greater than an average or threshold value are identified as preferred tissues or organs for providing hMSCs for allogenic transplant.

B. Methods for Assessing hMSCs Transplanted In Vivo or Ex Vivo

In some forms, the methods also include one or more steps for characterizing hMSCs administered to the patient during the course of the therapy or after the completion thereof. The characterization can include determining whether the hMSCs continue to express CD55, show increased or reduced expression of CD55, show reduced or increased surface deposition of complement, such as factor C3b, and monitoring the number and viability of hMSCs at time intervals following administration in vivo. For example, in some forms, samples of cells or tissues from transplant recipient patients are characterized prior to and following administration of hMSCs and optionally administration of one or more additional active agents, and/or other therapeutic interventions (e.g., radiation, cryoablation, surgery, etc.), in order to determine if one or more of the markers of disease, or the expression patterns of CD55 in hMSCs have changed. Therefore, in some forms, the methods include one or more steps for detecting and monitoring one or more biomarkers of a disease or disorder, or monitoring the number or expression products of hMSCs (including improved hMSCs) in the recipient subject. Suitable methods of detection are known in the art. For example, in specific forms, the methods include a step of contacting the hMSCs with a molecule that immuno-specifically or physio-specifically binds one or more proteins at the surface of the hMSCs, such as CD55 or C3b in vivo, or examining RNA expression for CD55 on hMSCs in a sample from a subject ex vivo, for example, using qPCR, microarray methods, or RNA-Seq. In some preferred forms, the methods employ one or more steps for administering and detecting a diagnostic or imaging molecule that immuno-specifically or physio-specifically binds Cd55 or C3b proteins at the surface of hMSCs. In some forms, the molecule that immuno-specifically or physio-specifically binds CD55 or C3b proteins is an antibody or an antigen-binding fragment thereof. In a preferred form, the antibody or antigen-binding fragment thereof is specific for the extracellular component of CD55.

C. Methods of Improving hMSCs

Methods of preparing improved hMSCs having increased expression of CD55 mRNA and/or increased surface presentation of CD55 are described. Typically, the methods include one or more steps of obtaining hMSCs, culturing the hMSCs in vivo in the presence of one or more active agents that stimulates or enhances the expression of CD55 mRNA and/or increases the surface expression of CD55 in the hMSC to provide improved hMSCs, and detecting the expression or surface presentation of CD55 in the improved hMSCs. In some forms, the methods include one or more steps for purifying or isolating the improved hMSCs.

In preferred forms, methods for providing improved hMSCs include exposing hMSCs to an active agent that functions as an HMG-CoA reductase inhibitor or an EGFR inhibitor. For example, in some forms, the methods contact hMSCs with one or more HMG-CoA reductase inhibitors, such as Atorvastatin (LIPITOR®), Fluvastatin (LESCOL®, LESCOL XL®), Lovastatin (MEVACOR®, ALTOPREV®), Pravastatin (PRAVACHOL®), Rosuvastatin (CRESTOR®), Simvastatin (ZOCOR®), or Pitavastatin (LIVALO®). In a preferred form, methods for providing improved hMSCs include contacting hMSCs with the HMG-CoA reductase inhibitor Atorvastatin. For example, in some forms, the methods include atorvastatin within culture media at an amount between 1 μM and 100 μM, for example, between about 2 μM and 50 μM, inclusive, for example, between 5 μM and 25 μM, inclusive. Typically, the hMSCs are cultured with atorvastatin for a time between one hour and one month, inclusive, for example, between one hour and 24 hours, inclusive, between one day and three weeks, inclusive, between one day and one week, inclusive, and between one week and two weeks, inclusive. In other forms, the methods contact hMSCs with one or more EGRF inhibitors, such as erlotinib, afatinib, brigatinib, icotinib, osimertinib, neratinib, gefitinib, cetuximab, panitumumab, lapatinib, dacomitinib, necitumumab and vandetanib. In a preferred form, methods for providing improved hMSCs include contacting hMSCs with the EGRF inhibitor erlotinib. For example, in some forms, the methods include Erlotinib within culture media at an amount between 1 μM and 100 μM, inclusive, for example, between about 2 μM and 20 μM, inclusive, for example, between 5 μM and 10 μM, inclusive. Typically, the hMSCs are cultured with erlotinib for a time between one hour and one month, inclusive, for example, between one hour and 24 hours, inclusive, between one day and three weeks, inclusive, between one day and one week, inclusive, and between one week and two weeks, inclusive.

Typically, the methods detect and/or quantitate CD55 expression in the improved hMSCs at a time following exposure to one or more active agents, for example, between about one hour and 12 hours, inclusive, or after 12 hours, for example, 24 hours, 36 hours, 48 hours, 60 hours or 72 hours or more than 72 hours after exposure to the agent(s). The methods assess the efficacy of the increased CD55 expression of the improved hMSCs and, in some forms, perform one or more rounds of selection to isolate the hMSCs having the highest expression of CD55.

D. Methods of Treatment and Dosage Regimes

In some forms, methods for selecting hMSCs and/or providing improved hMSCs include administering the selected and/or improved hMSCs to a recipient subject in need thereof for treatment or prevention of a disease or disorder.

Therapeutic regimens of improved hMSCs typically include treatment of a disease or symptom thereof, or a method for achieving a desired physiological change, including administering to a human being an effective amount of the improved hMSCs, optionally in combination with an additional active agent to treat the disease or symptom thereof, or to produce the physiological change.

Methods for therapeutic regimens including administering MSCs to a subject are well known in the art (see, for example, Ullah, et al., Bioscience reports, vol. 35,2 e00191. 28 Apr. 2015, doi:10.1042/BSR20150025, the contents if which are incorporated by reference herein). The improved hMSCs described herein can be utilized in a therapeutic regimen in the place of other hMSCs. For example, in some forms, the methods administer improved hMSCs to a recipient subject for the treatment or prevention of a chronic or acute disease or disorder in the subject. Exemplary diseases include acute graft versus host disease (GvHD) resulting from treatment of a cardiovascular disease, neurodegenerative disease and autoimmune disease. Typically, the methods administer an effective amount of improved hMSCs, and optionally one or more additional active agents as a single unit dosage (e.g., as dosage unit), or multiple sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

In some forms, the amount of the improved hMSCs is effective to alter a measurable biochemical or physiological marker associated with a disease or disorder in the recipient subject without development of, or with reduced development of GvHD in the recipient subject.

1. Diseases to be Treated

The methods administer improved hMSCs to prevent acute graft versus host disease (GvHD). The improved hMSCs are also employed to treat or prevent one or more diseases such as neurodegenerative diseases and autoimmune diseases. Exemplary diseases that can be treated using the described improved hMSCs include all diseases that can be treated using normal or non-improved hMSCs, such as neurodegenerative diseases including Amylotrophic lateral sclerosis (AML), Parkinson's disease (PD), muscular dystrophy (MD), Alzheimer disease (AD), autoimmune diseases including Rheumatoid arthritis (RA), Chron's Disease (CD), ulcerative colitis and Type 1 diabetes, and cardiovascular diseases such as myocardial repair for myocardial infarction, ischemic stroke therapy, and heart failure secondary to left ventricular injury, as well as other diseases and disorders such as liver disorders, respiratory disorders, spinal cord injury, kidney failure, skin diseases, aplastic anemia, systemic sclerosis, and Osteogenesis imperfecta.

a. Graft Versus Host Disease (GvHD)

In a preferred form, the methods administer improved MSCs for prevention of one or more symptoms of graft versus host disease (GVHD). GvHD is a syndrome, characterized by inflammation in different organs. GvHD is commonly associated with bone marrow transplants and stem cell transplants. In GvHD, donated bone marrow or stem cells view the recipient's body as foreign, and the donated cells/bone marrow attack the body.

Solid organ transplant-associated GvHD is an infrequent and potentially lethal complication. In some forms, the GVHD is associated with solid organ transplantation, e.g., kidney transplant and liver transplant.

The two types of GvHD are acute and chronic: acute graft-versus-host-disease is characterized by selective damage to the liver, skin (rash), mucosa, and the gastrointestinal tract, as well as the immune system (the hematopoietic system, e.g., the bone marrow and the thymus) itself, and the lungs in the form of immune-mediated pneumonitis. Biomarkers can be used to identify specific causes of GvHD, such as elafin (also known as peptidase inhibitor-3, skin-derived antileukoproteinase or trappin-2) in the skin; chronic graft-versus-host-disease also effects the liver, skin (rash), mucosa, and the gastrointestinal tract, as well as the immune system and the lungs, but over its long-term course can also cause damage to the connective tissue and exocrine glands.

i. Acute GvHD

In some forms, the methods administer improved HSCs prevent acute GvHD in a recipient subject. The acute or fulminant form of aGvHD is normally observed within the first 10 to 100 days post-transplant and is present in about one-third to one-half of allogeneic transplant recipients. Symptoms include a rash, burning, and redness of the skin on the palms and soles, nausea, intestinal inflammation, sloughing of the mucosal membrane, vomiting, stomach cramps, diarrhea (watery and sometimes bloody), loss of appetite, jaundice, abdominal pain, and weight loss. Therefore, in some forms, the methods administer improved hMSCs to prevent one or more symptoms of acute GvHD in a recipient subject, including rash, burning, and redness of the skin on the palms and soles, nausea, intestinal inflammation, sloughing of the mucosal membrane, vomiting, stomach cramps, diarrhea (watery and sometimes bloody), loss of appetite, jaundice, abdominal pain, and weight loss. Methods for detecting and monitoring acute GvHD in a subject are known in the art. For example, GvHD of the GI tract is typically diagnosed via intestinal biopsy. Liver GvHD is measured by the bilirubin level in acute patients. Skin GvHD results in a diffuse red maculopapular rash, sometimes in a lacy pattern.

Acute GvHD is staged as follows: overall grade (skin-liver-gut) with each organ staged individually from a low of 1 to a high of 4. Patients with grade IV GvHD usually have a poor prognosis. If the GvHD is severe and requires intense immunosuppression involving steroids and additional agents to get under control, the patient may develop severe infections as a result of the immunosuppression and may die of infection. Therefore, the methods can prevent GvHD or reduce the grade of GvHD in a recipient patient as compared to a control patient who did not receive the improved hMSCs. For example, in some forms, the administration of improved hMSCs reduces the grade of GvHD in an organ of a recipient subject by between one and four points.

### IV. Kits

Kits are also disclosed. The kits can include, for example, a dosage supply of an active agent, such as a EGRF inhibitor and/or an HMG-CoA reductase inhibitor, or a combination thereof, and one or more reagents for isolating and culturing hMSCs in vitro. In some forms, kits also include a plurality live, viable hMSCs, for example, in a frozen state or in another state suitable for distribution of live, viable cells. The active agents can be supplied alone (e.g., lyophilized), or in a pharmaceutical composition. The active agents can be in a unit dosage, or in a stock that should be diluted prior to administration. In some forms, the kit includes a supply of one or more pharmaceutically acceptable carriers. The kit can also include devices for administration of the active agents or compositions, for example, syringes. The kits can include printed instructions for administering the compound in a use as described above.

The present invention is further understood by reference to the following non-limiting examples.

### EXAMPLES

### Example 1: Bioinformatic Analysis of hMSCs from Different Sources

Strategy

A comprehensive biological analysis was performed to compare three types of hMSCs to explore the differences in their profiles. RNA sequencing enhances the analysis of whole transcriptomes collectively, allowing a comprehensive annotation and quantification of a vast number of genes in a single run.

Materials and Methods

To better understand the molecular differences among different the three types of hMSCs, adipose tissue, bone marrow and umbilical cord were collected from different normal individuals, and their respective transcriptome was examined by RNA sequencing. An average of 30 million 150-base long reads from MSCs derived from adipose tissue, bone marrow and umbilical cord tissue. These reads were aligned to the human reference genome by STAR, which generated an expression level for each gene.

Next the dataset was examined together with three publicly available transcriptomic datasets involving AT-, UC- vs BM-hMSC comparisons.

Results

Hierarchical clustering and principal components analysis (PCA) showed that overall gene expressions among biological replicates were very similar, consistent with a high experimental reproducibility. On the other hand, AT-, BM- and UC-hMSCs could be separated into different clusters, confirming that each type of hMSCs had their own distinct transcriptomic profiles.

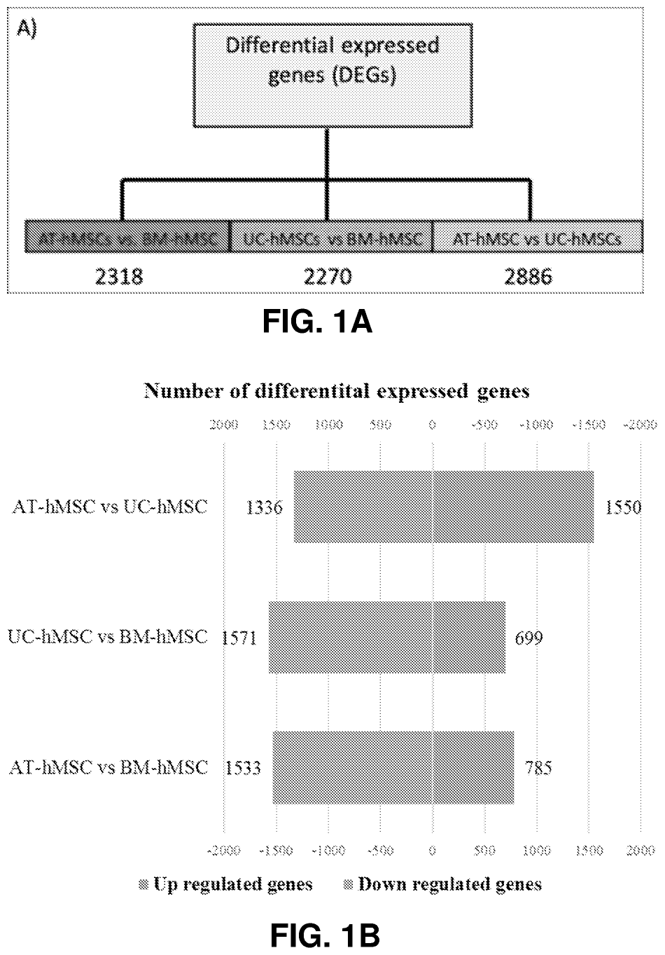

A comparison between AT- vs BM, AT- vs UC and BM vs UC showed that more than 2000 genes were differentially expressed by more than 1-fold and p-value less than 0.05 for each comparison (FIG. 1A), the number of up and down-regulated gene have been separated for further analysis (FIG. 1B).

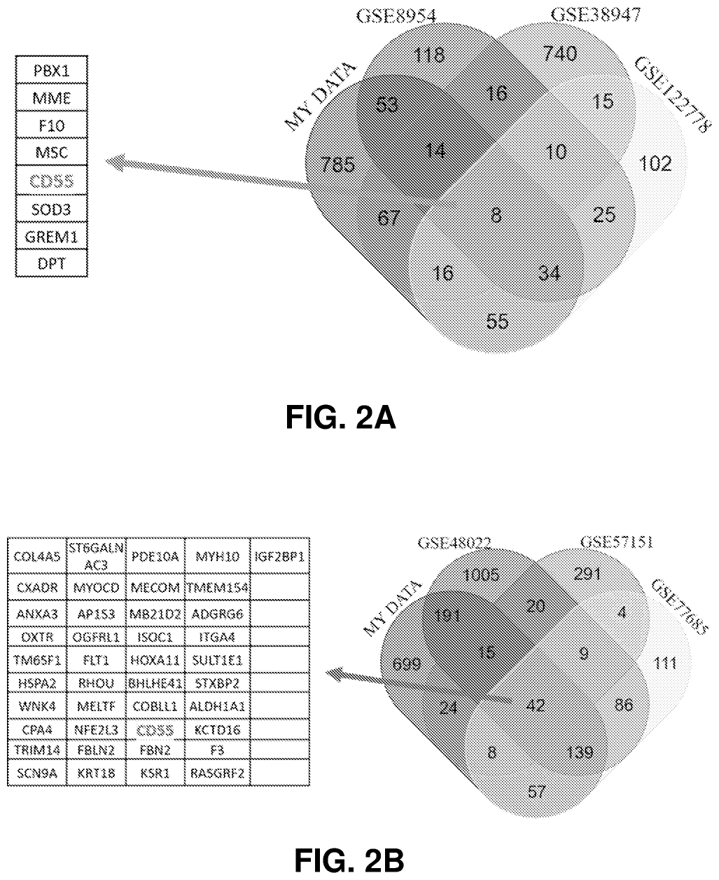

Venn diagrams of differentially expressed genes from our dataset and three different datasets revealed that 8 and 42 genes were up-regulated in AT- and UC-hMSCs compared to BM-hMSCs and 42 genes were up-regulated respectively in UC- versus BM-hMSCs respectively in all datasets (FIGS. 2A-2B). Among these datasets, CD55, also known as complement decay-accelerating factor (DAF), was the only gene upregulated in both AT-hMSCs and UC-hMSCs compared to BM-hMSCs. CD55 is a regulator of complement response that prevents amplification of the complement cascade and protects against complement-mediated cell lysis.

### Example 2: CD55 is Differentially Expressed in Different hMSC Lineages

Materials and Methods

Since it was found that CD55 was the only regulated gene from the Venn diagram of all datasets, the GO and KEGG enrichment analysis was examined of two public datasets from AT-hMSC versus BM-hMSC. The expression of CD55 in hMSC was then validated by RT-qPCR and flow cytometry.

Results

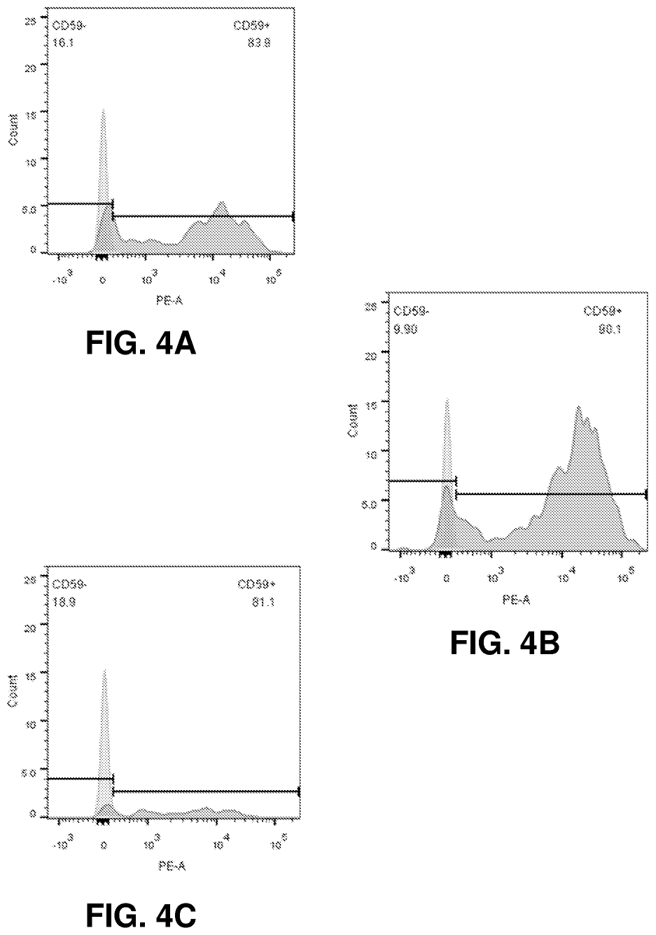

Results showed that up-regulated gene in AT-hMSC was involved in the complement response and negatively regulated inflammatory response. It was the top differentially expressed gene sets AT and BM-hMSCs. Furthermore, we revealed the expression of complement pathway related gene in the dataset, heatmap expression showed that CD55 was downregulated in BM-hMSC compared to AT-hMSC and UC-hMSC, whereas the other two complement regulatory genes CD46 and CD59 were expressed similarly. RT-qPCR results showed that CD55 mRNA is more abundant in AT-hMSCs than UC-hMSCs and BM-hMSCs with the fold differences of AT-hMSC vs BM-hMSC: 4.9; AT-hMSC vs UB-hMSC:2.4 (FIG. 3A). Moreover, flow analysis showed that a greater proportion of AT-hMSCs (83.42±9.57%) and UC-hMSC (63.4±7.87%) expressed CD55 on the cell surface, and at higher levels, than BM-hMSCs (14.46±4.63%) (FIG. 3B). Conversely, the expression of CD59, another complement regulatory protein, was detected in >80% of hMSCs at similar levels among BM-, AT- and UC-hMSCs (FIGS. 4A-4C).

### Example 3: Adipose Tissue-Derived hMSCs have Increased Expression of CD55 and are Less Sensitive to Complement-Induced Injury

To assess whether increased CD55 expression on the cell surface of AT-hMSCs may inhibit complement induced damage and hence acquire cell survival advantage as compared to BMs- and UC-hMSCs.

Methods

To evaluate the interaction between hMSCs and complement proteins in serum, we incubated BM-, AT- and UC-hMSCs with mouse serum.

C3b is crucial for the formation of the membrane attack complex, which induces membrane rupture, thus lower levels of C3b would render AT-hMSCs less prone to cell damage by complement activation. We tested this with the membrane permeant BCECF ester.

Moreover, we examined the relationship between C3 deposition and the positive population of CD55 on hMSCs by plotting the correlation curve.

Apart from that, we also performed an immunofluorescence staining on tissue sections on day 21.

Results

Significantly reduced deposition of the complement activation product C3b was found on the cell surface of AT-hMSCs (3.57±2.66%) relative to UC-hMSCs (9.255±4.69%) and BM-hMSCs (23.525±3.75%), as determined by flow cytometry (FIG. 5A).

Upon intracellular hydrolysis, the dye becomes impermeant, thus its release is used to monitor cell membrane integrity. hMSCs were loaded with BCECF ester, exposed them to serum and monitored the release of this dye. Consistent with a hypothesis, significantly less cytotoxicity was detected in the supernatant of AT-hMSC cultures (6.85±1.02%, BM-hMSC 23.53±2.67%, UC-hMSC 12.44±2.42%) indicating reduced membrane leakage from these cells (FIG. 5B). Blocking CD55 by pre-incubating hMSCs with an antibody against this protein significantly increased C3b deposition on AT-hMSC cells from 3.57 to 90.2%.

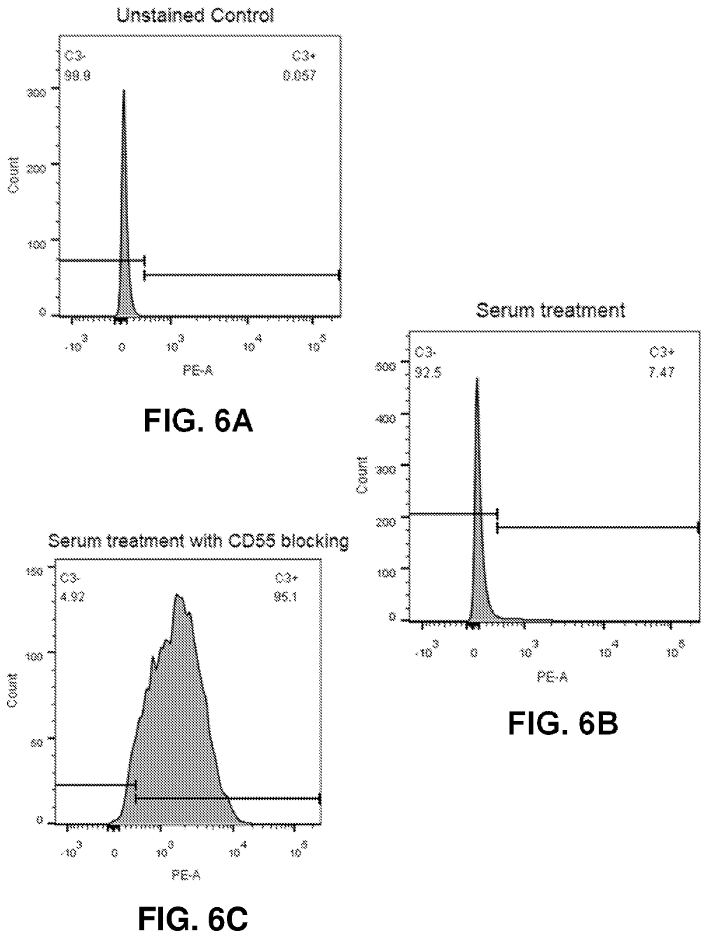

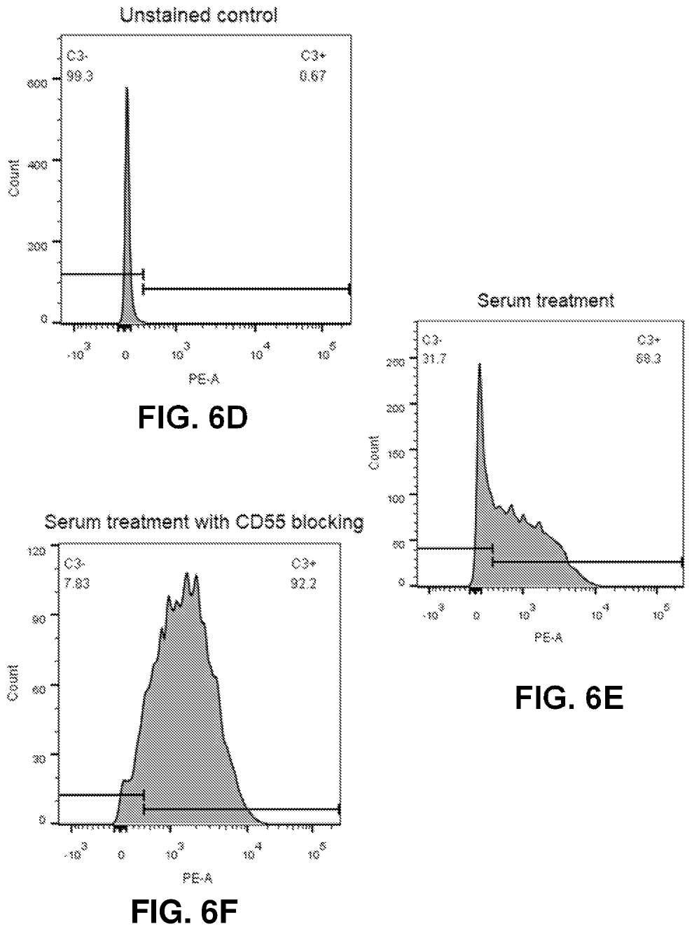

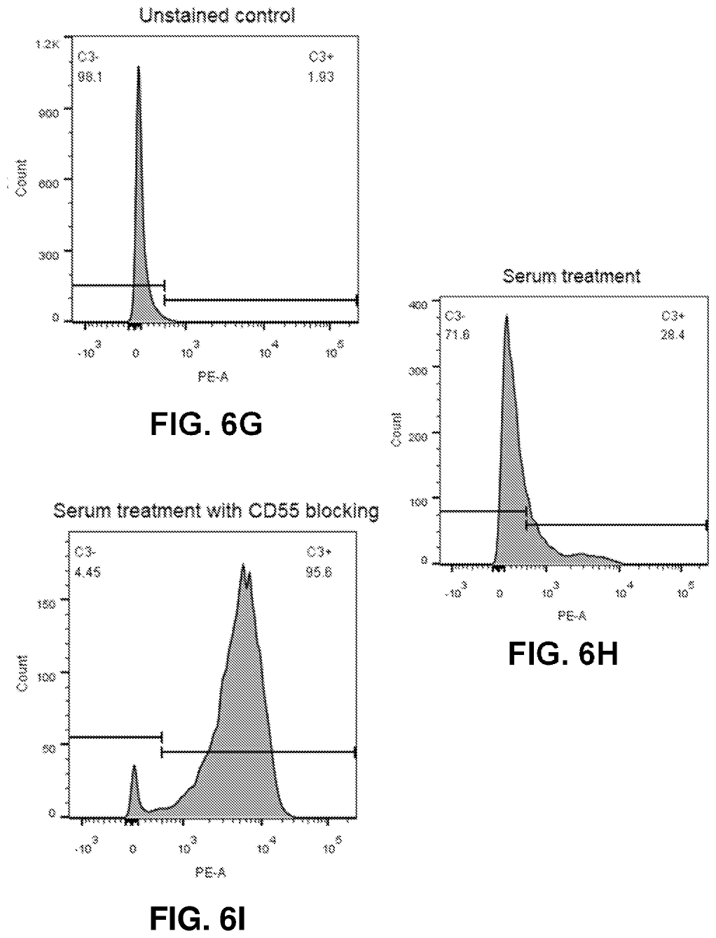

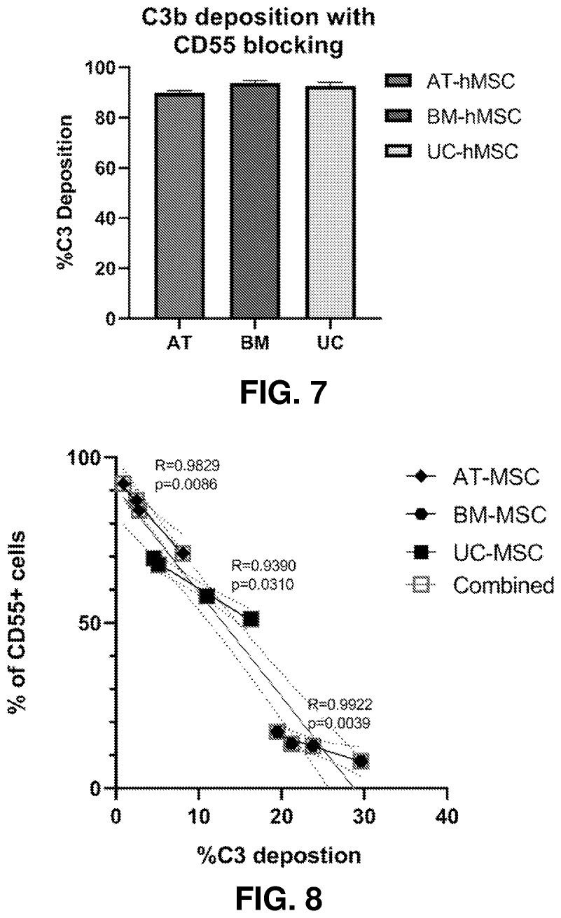

By plotting the correlation curve of C3 deposition and the positive population of CD55 on hMSCs, results showed that there was negative correlation between positive % of CD55 cell and C3 deposition (FIGS. 6A-6I, 7). These suggested that when the % of CD55 on hMSCs increases, C3 deposition decreases, implying the effect of CD55 on complement inhibition. Our results support the role of CD55 in the suppression of complement activation. In summary, we showed that AT-hMSCs were much less sensitive to complement induced injury and this was partially correlated with the increased expression of CD55.

At day 21 post transplantation, the expression of C3 on liver and colon from BM-hMSC and UC-hMSC were highly expressed which is similar to positive control group, whereas AT-hMSC and negative control group did not express any C3 on the tissues (FIG. 8). For the C3 expression of lung at day 21 post transplantation, positive control group was highly expressed, whereas the hMSC treatment groups were slightly expressed. These results further confirmed that the inhibition of complement activation by AT-hMSC is superior to the other hMSCs.

### Example 4: Treatment of Erlotinib and Atorvastatin onto hMSCs Resulted in Significantly Increased of CD55 Expression in hMSCs

Since the expression of CD55 could inhibit C3 deposition, which led to inhibition of complement cell lysis whether there are any chemicals or drugs that could increase the expression of CD55 in hMSCs was investigated. Such methods can potentially enhance the complement inhibitory effect of hMSCs.

Methods

We performed a co-expression analysis of DEGs by calculated the correlation value between AT-hMSC vs BM-hMSC and AT-hMSC vs UC-hMSC, then we selected the genes that are within 0.75 and 1 correlation value and submitted to The Database for Annotation, Visualization and Integrated Discovery (DAVID) for analysis.

hMSC were treated with Erlotinib, which is an EGFR inhibitor, on different dosages within 24 hours,

Results

Results showed that AT-hMSC has more genes expressed in EGFR pathway then the other two types of MSCs.

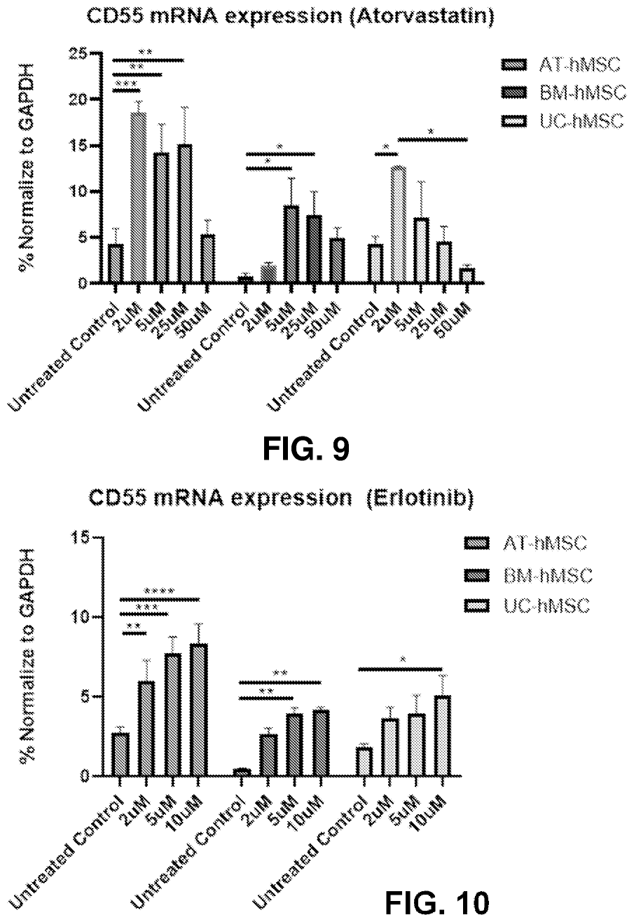

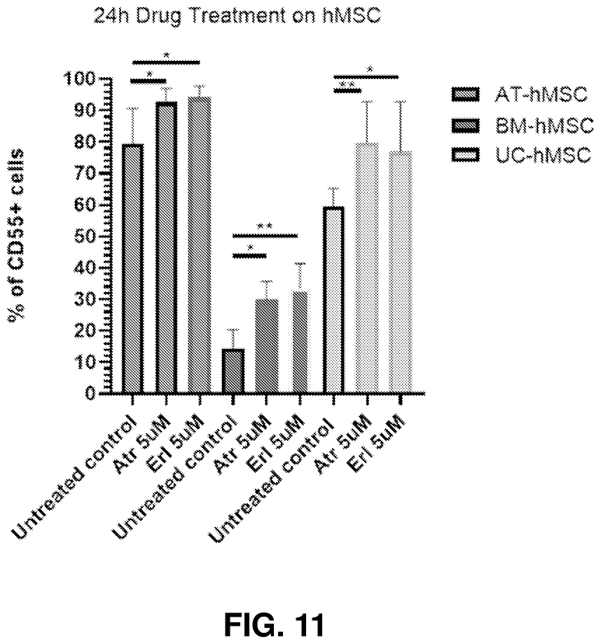

All hMSCs were found to have increased CD55 expression with significance difference in mRNA level compared to untreated control in a dose dependent manner (FIGS. 9-10), surface protein level of CD55 were also significantly upregulated compared with untreated control as assessed by flow cytometry (FIG. 11).

The effect of atorvastatin on hMSCs was also assessed. Results showed that atorvastatin could also increase the expression of CD55 of hMSCs in both mRNA and protein level (FIGS. 9-11). However, there was not a similar dose dependent relationship as in EGFR inhibitor treatment. It appeared that atorvastatin has a relatively narrow therapeutic window and after a relatively low dose (2 to 5 μM), the response seems to be lower with higher dosage.

Summary

It has been demonstrated that CD55 is highly expressed in AT-hMSC and UC-hMSC compared to BM-hMSC based on RNA-seq results. It was further established that Adipose Tissue-derived hMSC have the highest expression of CD55 in terms of mRNA and surface protein level among all hMSCs. This CD55 expression correlated to relatively low C3 deposition and therefore will be less affected by complement lysis. AT-hMSC has the lowest C3 deposition and cytotoxicity among all hMSCs upon treatment with mouse serum. Moreover, we also found that AT-hMSC could suppressed C3 deposition on colon and liver at day 21 post transplantation by immunofluorescence staining. These results suggested that AT-hMSC have a higher inhibitory effect on complement activation as compared to other hMSCs. Clinical application of hMSCs required several characteristics to be considered as optimal source. One of them is the in vitro expandability, it is important to examine the proliferate rate of each source of hMSCs to access the potential of expansion. AT-hMSC were showed to have the highest proliferation rate, as confirmed by XTT proliferation assay. Moreover, the doubling time of BM-hMSC was 2-fold greater than AT-hMSC, whereas UC-hMSC was intermediate to those of BM-hMSC and AD-hMSC.

The population of senescent cells were also identified at different passages. BM-hMSC and UC-hMSC were found to have a large population of senescent cells at passage 12, whereas AT-hMSC only showed a little population of senescent cells. Apart from the phenotype, cell cycle marker genes such as Cyclin D2, CDK2, CD4 of AT-hMSC were found significantly upregulated as compared to BM-hMSC and UC-hMSC. These marker genes have an important function on DNA replication, which are actively involved in G1 and S phase of the cell cycle. The senescence marker gene like P16 and P21 were also found to be significantly down-regulated in AT-hMSC compared with BM-hMSC and UC-hMSC. AT-hMSCs likely have a higher proliferation rate compared with BM-hMSC and UC-hMSC. Together, the results suggested that AT-hMSC are more favorable than BM-hMSC and UC-hMSC in terms of the in vitro expandability.

Furthermore, hMSCs have been shown to suppress multiple components of the immune system relevant to the pathogenesis of aGVHD. In this study, we showed that BM, AT, and UC-derived hMSCs could all significantly modulate dendritic cell activation, T-helper cell differentiation, and T-reg proliferation in vitro. While some differences were detected among the BM-, AT and UC-hMSCs, they were either not statistically significant, or were modest. The only statistically significant difference among the three types of hMSCs was the induction of Th2-mediated IL4 secretion, where BM-hMSCs was more effective.

### Example 5: AT-hMSC Treatment had the Highest Survival Rate in a Mouse Model of Acute Graft Versus Host Disease

Although there are many studies on using hMSC as cellular therapy for aGVHD, its mechanism remains not fully explored, thus an established murine model of GVHD was used to study the immunomodulatory efficacy of hMSCs.

Methods

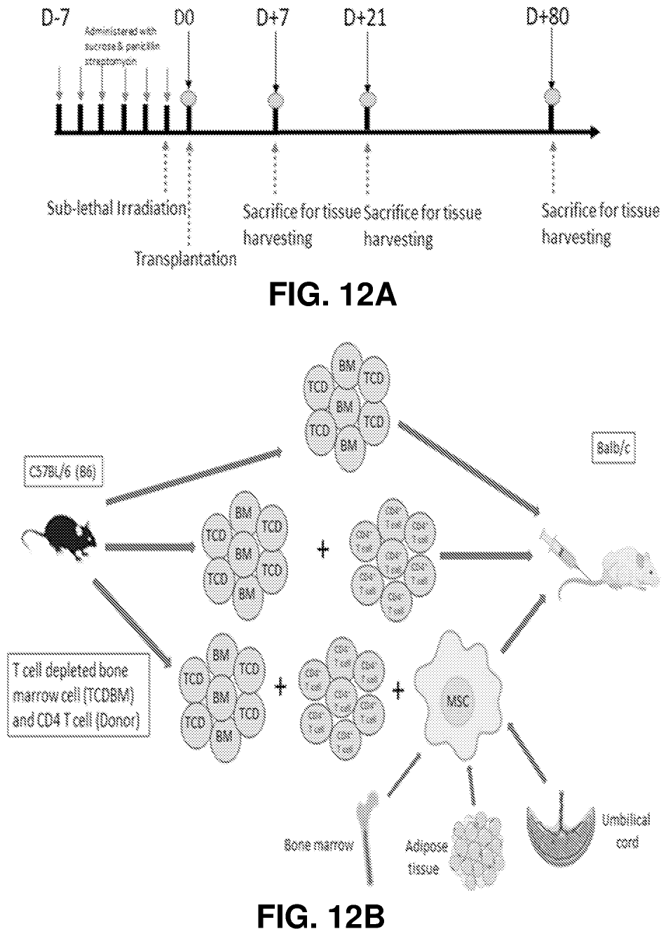

The murine model of GVHD is first initiated by the irradiation condition regimen on the recipient mice, whereas BALB/C mice as recipient mice and C57BL/6 as donor mice, were injected with or without hMSC (FIGS. 12A-12B). Acute GVHD symptoms were confirmed by phenotypic changes such as loss of fur, hunching and diarrhea from the positive control group.

Results

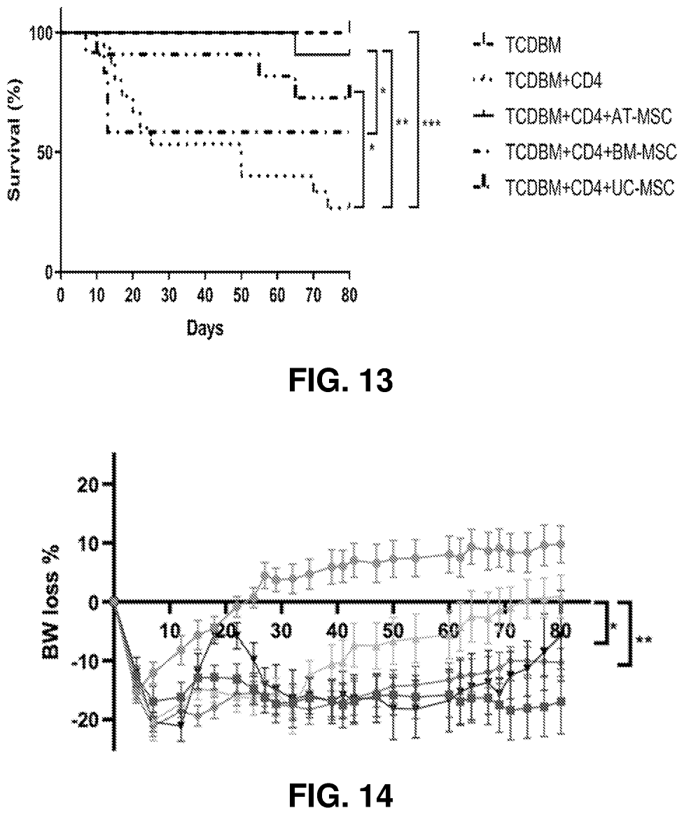

Among the hMSCs treatment group, AT-hMSC treatment group had the highest survival rate with less aGVHD phenotype compared with other hMSCs treatment group. BM-hMSCs group showed severe loss of fur from the mice. The survival rate of the positive control group mice at day 21 is around 50%, indicated the severity and the progression of aGVHD in our diseased model (FIG. 13). Body weight loss is also a crucial clinical magnification on studying aGVHD (FIG. 14). In our study, all group exhibited a weight loss in the first week post transplantation, this weight change was probably due to the adverse effect of conditional regimen. Mice received AT-hMSC started to recover their body weight at 21 days post transplantation, whereas the weight of BM-hMSC and UC-hMSC treatment group remained at a lower level up to day 80. Besides, we also observed that there were histopathological changes in our model, scored according to Table 1.

TABLE 1
Scoring for changes in murine acute GVHRD model

| Parameter | Animal ID | Score |
|---|---|---|
| Weight loss | <10% | 0 |
| | 10-25% | 1 |
| | >25% | 2 |
| Posture | Normal | 0 |
| | Hunching noted only at rest | 1 |
| | Severe hunching | 2 |
| Activity | Normal | 0 |
| | Mild to moderately decreased | 1 |
| | Stationary unless stimulated | 2 |
| Fur texture | Normal | 0 |
| | Normal Mild to moderate ruffling | 1 |
| | Severe ruffling/poor grooming stimulated | 2 |
| Skin integrity | Normal | 0 |
| | Scaling of paws/tail | 1 |
| | Obvious areas of denuded skin | 2 |
| | Total | 0-10 |

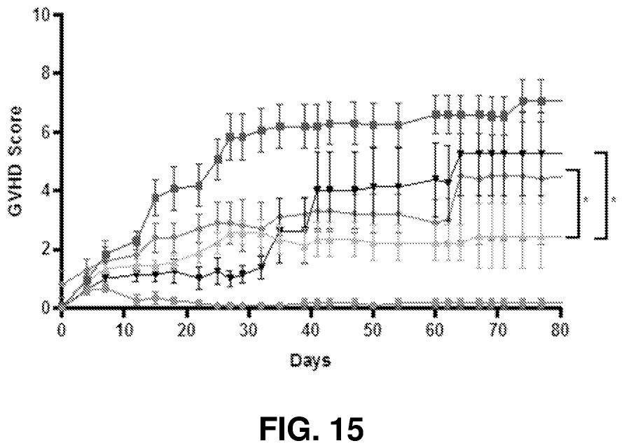

High number of lymphocyte infiltration was found in the positive control group mice, suggesting severe aGVHD occurred and was responsible for the persistent weight loss and death. Of the hMSC treatment group, AT-hMSC seems to be the best treatment group to ameliorate aGVHD, as the mice retained a normal morphology of colon, liver and spleen at day 7 and 21 post transplantation, whereas those of BM-hMSC and UC-hMSC have shown loss of crypt in colon and a large amount of lymphocyte infiltration in colon and liver, survivor of BM-hMSC treatment mice even developed cGVHD symptoms in liver such as portal fibrosis (FIG. 15).

Summary

The results are consistent with previous publications, which reported hMSCs could suppress T-cell proliferation and secretion of pro-inflammatory cytokines. Most studies used BM hMSCs for these immune functional studies. The results of this study showed that AT-hMSCs were more potently against aGVHD in vivo than UC- or BM-hMSCs. A recent report by Gregoire et al showed that AT-, UC-, and BM-hMSCs all failed to significantly increase overall survival of mice with aGVHD. However, hMSCs were used as salvage treatment in that study and infusion was withheld until symptoms appeared. However, our hMSCs injected early as prophylaxis to prevent aGVHD. These data showed that both AT-, UC-hMSCs could protect the mice against aGVHD in vivo.

### Example 6: AT-hMSC Effectively Suppressed the Expression of Chemokine Receptor

Many studies suggested that there is an up-regulation of chemokine receptors from the acute GHVD target organs, the distribution of the chemokine profile was different in each target organs. Of these receptors, CCL3 and CXCL9 are the chemokine ligands that are found in all aGVHD target organs such as liver, gut, and skin.

The increased expression of CCL3 and CXCL9 are suggested to facilitate the migration of T cell into aGVHD target organs. The expression of CCL3 and CXCL9 was initially upregulated to facilitate the migration of donor cell, which also include lymphoid organs. The data suggested that in the later stage of around day 7 and day 21 post transplantation, hMSC were able to suppress the expression of chemokine receptors and inhibit the migration of T cell lymphocytes to target organs, and as a result ameliorate the development of aGVHD. AT-hMSC suppressed the expression chemokine receptor most effectively in colon and liver at day 7 and 21 post transplantation.

While immunosuppressive properties may be important for the use of hMSCs in immune disorders, proliferation and engraftment properties are relevant for all applications of hMSCs in cell therapy. hMSCs are thought to be cleared rapidly after transplantation, but most of these studies were done using BM-hMSCs. Here we demonstrated the presence of hMSCs in the host tissues was done via in vivo imaging. CM-Dil labeled hMSCs were recognized in the liver, colon, lung, and spleen at 7 days and 21 days after transplantation. CM-Dil was selected for labeling cell because it has been shown to have a long-lasting effect up to 1 month, but AT-hMSCs exhibited better engraftment in the liver, lung and liver, and this may underlie the higher survival rate of this treatment group. Takahashi recently showed that murine AT-MSCs engrafted better in injured spinal cord, and this was associated with better preservation of axons and enhanced vascularization. Consistent with the superior in vivo efficacy against aGVHD as we reported here. AT-MSCs have been shown to be as good as BM-MSCs at treating Crohn's disease in mice or more effective in animal models with a broad range of diseases including system sclerosis, ischemic stroke therapy, myocardial infarction, and spinal injury.

Summary

The overall gene expression profiles of hMSC were studied by RNA-sequencing techniques, and differential gene expression was determined in relation to BM-hMSCs. GO and KEGG analysis has demonstrated a consistent upregulation of well-characterized genes in complement and coagulation cascade, negative regulation of inflammatory response from AT-hMSC versus BM-hMSC. Besides, GSEA analysis of the DEGs of AT-hMSC and BM-hMSC also showed that AT-hMSC could suppress the pathway of 1) allograft rejection; and 2) Th1 Th2 T cell differentiation. These results suggested that AT-hMSC may have a particular function to suppress the rejection from the host immune system. We therefore focused on these genes and their role in hMSC. Among these, CD55 was differentially upregulated in AT-hMSC and UC-hMSC compared to BM-hMSC. CD55, also called decay-accelerating factor (DAF), is a single chain membrane bound regulatory protein that modulate the complement system. The expression of CD55 in AT-hMSCs was found to be significantly higher than BM-hMSC and UC-hMSC in terms of mRNA and surface protein level, suggested that AT-hMSC may have a better complement inhibitory effect then other hMSCs.

Many studies showed that MSCs are immune-privileged and can partially evade the monitor of host immune cells. Owing to these advantages, most of the hMSCs in clinical trials are from allogeneic donors. However, it has been reported that infused hMSCs can be recognized and damaged by the complement system after infusion of hMSC, therefore reduced therapeutic efficacy of hMSC transplantation. A crucial finding is that contact of hMSCs to serum activated complement could damage the hMSCs. Despite of the fact that hMSCs expressed the three surface complement regulators (CD46, CD55 and CD59), they could still activate the complement system when treated with serum and resulted in cell cytotoxicity. Thus, the survival of hMSCs is considerably reduced after IV infusion, even though their potent immunosuppressive activity has been demonstrated in vitro. It was found that AT-hMSC has a better effect of complement inhibition upon treatment with serum compared with other two types of hMSCs. Here, it has been established that there are two existing drugs which can enhance the expression of CD55 in hMSC. This finding may supplement the current hMSC-based clinical trials that have been unsatisfactory.

Although there are many registered hMSC-based clinical trials in aGVHD, no conclusion to suggest which sources of hMSC are the most effective sources on treating aGVHD up to now. The short lifespan of hMSCs after administration has been described as a “hit-and-run” theory. It is speculated that, even if hMSCs only survive for a short period of time after administration, this limited capacity is already very useful in treated patients. This theory is getting more attention in the field, indicates that just as a slight increase in hMSC survival and engraftment after infusion could have a large influence on hMSCs treatment. It was demonstrated that after treatment of erlotinib and atorvastatin onto hMSCs resulted in significantly increased of CD55 expression in hMSCs, suggesting that these cells may be more resistant to cell lysis upon complement activation. Adding these drugs onto the hMSCs before administration will improve hMSC viability and function and provide a better outcome for future hMSC-based therapies.

### CONCLUSIONS

Previous studies have demonstrated that BM-, AT-, and UC-hMSCs have different biological characteristics, but direct comparisons of in vivo efficacy are lacking and have not been addressed in aGVHD models. These data correlate the in vitro properties of hMSCs generated from different tissues with their abilities to suppress aGVHD in vivo. Here, it is demonstrated that hMSCs could be successfully derived from AT, BM and UC. They all modulate multiple components of the immune system in vitro and protect the mice against aGVHD. However, AT-hMSCs had better proliferative potential, and were more effective in maintaining survival. AT-hMSCs also ameliorated the clinical signs of aGVHD better than BM- and UC-hMSCs. It is therefore concluded that AT is a superior cell source of hMSCs for the treatment of acute GVHD.

This study focused on acute GVHD, but hMSCs are currently being tested in clinical trials for the prevention and treatment of a range of disorders involving the dysregulation of the immune system e.g., acute respiratory distress syndrome associated with COVID-19, Crohn's disease, Inflammatory Bowel Disease etc. The data are helpful in broadening the clinical application of AT-hMSCs as a form of cell therapy in the future.

The invention will be further understood by virtue of the following numbered paragraphs.

1. A method comprising culturing a human mesenchymal stem cell (hMSC) with culture media comprising one or more active agents that induce or stimulate expression of CD55 mRNA and/or increase expression of CD55 on the surface of the hMSC,

whereby the incubated hMSC has increased expression of CD55 mRNA and/or increased expression of CD55 on the surface of the cultured hMSC as compared to a control hMSC similarly cultured in similar culture media but lacking the one or more active agents.

2. The method of paragraph 1, wherein the active agent is an EGRF inhibitor or an HMG-CoA reductase inhibitor.

3. The method of paragraph 2, wherein the HMG-CoA reductase inhibitor is selected from the group consisting of atorvastatin, fluvastatin, lovastatin, pravastatin, rosuvastatin, simvastatin, and pitavastatin.

4. The method of paragraph 2 or 3, wherein the HMG-CoA reductase inhibitor is atorvastatin.

5. The method of paragraph 4, wherein atorvastatin is present in culture media at a concentration between about 2 μM and 50 μM, inclusive, preferably between 5 μM and 25 μM, inclusive.

6. The method of paragraph 2, wherein the EGRF inhibitor is selected from the group consisting of erlotinib, afatinib, brigatinib, icotinib, osimertinib, neratinib, gefitinib, cetuximab, panitumumab, lapatinib, dacomitinib, necitumumab, and vandetanib.

7. The method of paragraph 2 or 6, wherein the EGRF inhibitor is erlotinib.

8. The method of paragraph 7, wherein erlotinib is present in culture media at a concentration between about 2 μM and 25 μM, inclusive, preferably between 5 μM and 10 μM, inclusive.

9. The method of any one of paragraphs 1-8 further comprising quantitating the level of CD55 expressed in the cultured hMSC.

10. A human mesenchymal stem cell (hMSC) prepared according to the method of any one of paragraphs 1-9.

11. A method of identifying a human mesenchymal stem cell (hMSC) for allogenic transplantation, the method comprising measuring the level of CD55 mRNA and/or the level of expression of CD55 on the surface of the hMSC, wherein the hMSC is identified as one suitable for transplantation if the level of CD55 mRNA and/or the level of expression of CD55 on the surface of the hMSC is higher than a minimum threshold of the level of CD55 mRNA and/or the level of expression of CD55 on the surface.

12. The method of paragraph 11, wherein the hMSC is isolated from a source selected from the group consisting of bone marrow, adipose tissue, amniotic fluid, endometrium, dental tissues, umbilical cord, Wharton's jelly, amniotic membrane, limb bud, menstrual blood, peripheral blood, placenta and fetal membrane, salivary gland, skin, foreskin, sub-amniotic umbilical cord lining membrane, and synovial fluid.

13. The method of paragraph 12, wherein the hMSC is isolated from adipose tissue.

14. The method of any one of paragraphs 11-13 further comprising administering the hMSC identified as one suitable for transplantation to a subject in need thereof.

15. The method of paragraph 14, wherein the subject is undergoing allogenic transplantation or is suffering from one or more symptoms associated with acute graft versus host disease.

16. The method of any one of paragraphs 11-15, wherein the minimum threshold is about 10%, 20%, 30%, 40%, 50%, 60%, 70%, 80%, or 90% of the level of CD55 mRNA and/or the level of expression of CD55 on BM-hMSCs.

17. A method comprising administering to a subject in need thereof an effective amount of human mesenchymal stem cells (hMSCs), having increased expression of CD55 mRNA and/or increased expression of CD55 on the surface of the hMSCs as compared to a minimum threshold of the level of CD55 mRNA and/or the level of expression of CD55,

wherein the subject is suffering from or at risk of developing one or more degenerative and/or immune disorders.

18. The method of paragraph 17, wherein the minimum threshold is at least 10%, 20%, 30%, 40%, 50%, 60%, 70%, 80%, or 90% of the level of CD55 mRNA and/or the level of expression of CD55 on BM-hMSCs.

19. The method of paragraph 17 or 18, wherein the hMSCs are isolated from adipose tissue.

20. The method of any one of paragraphs 17-19, wherein, prior to administering, the hMSCs are cultured with one or more active agents that induce or stimulate expression of CD55 mRNA and/or increase expression of CD55 on the surface of the hMSCs as compared to a control cell without the one or more active agents.

21. The method of paragraph 20, wherein the active agent is an EGRF inhibitor or an HMG-CoA reductase inhibitor.

22. The method of paragraph 21, wherein the HMG-CoA reductase inhibitor is selected from the group consisting of atorvastatin, fluvastatin, lovastatin, pravastatin, rosuvastatin, simvastatin, and pitavastatin.

23. The method of paragraph 21 or 22, wherein the HMG-CoA reductase inhibitor is atorvastatin.

24. The method of paragraph 21, wherein the EGRF inhibitor is selected from the group consisting of erlotinib, afatinib, brigatinib, icotinib, osimertinib, neratinib, gefitinib, cetuximab, panitumumab, lapatinib, dacomitinib, necitumumab, and vandetanib.

25. The method of paragraph 21 or 24, wherein the EGRF inhibitor is erlotinib.

26. The method of any one of paragraphs 17-25, wherein the one or more degenerative and/or immune disorders are one or more neurodegenerative diseases and/or autoimmune diseases.

27. The method of paragraph 26, wherein the one or more neurodegenerative diseases are selected from the group consisting of amyotrophic lateral sclerosis, Parkinson's disease, muscular dystrophy, and Alzheimer disease.

28. The method of paragraph 26, wherein the one or more autoimmune diseases are selected from the group consisting of rheumatoid arthritis, Crohn's disease, ulcerative colitis, and Type 1 diabetes.

29. The method of any one of paragraphs 17-25, wherein the degenerative and immune disorder is graft versus host disease (GvHD).

30. The method of paragraph 29, wherein the degenerative and immune disorder is acute GvHD.

We claim:

1. A method comprising culturing a human mesenchymal stem cell (hMSC) with culture media comprising one or more active agents that induce or stimulate expression of CD55 mRNA and/or increase expression of CD55 on the surface of the hMSC,
whereby the incubated hMSC has increased expression of CD55 mRNA and/or increased expression of CD55 on the surface of the cultured hMSC as compared to a control hMSC similarly cultured in similar culture media but lacking the active agents.

2. The method of claim 1, wherein the active agent is an EGRF inhibitor or an HMG-CoA reductase inhibitor.

3. The method of claim 2, wherein the HMG-CoA reductase inhibitor is selected from the group consisting of atorvastatin, fluvastatin, lovastatin, pravastatin, rosuvastatin, simvastatin, and pitavastatin.

4. The method of claim 3, wherein the HMG-CoA reductase inhibitor is atorvastatin.

5. The method of claim 4, wherein the atorvastatin is present in culture media at a concentration between about 2 μM and 50 μM, inclusive, preferably between 5 μM and 25 μM, inclusive.

6. The method of claim 2, wherein the EGRF inhibitor is selected from the group consisting of erlotinib, afatinib, brigatinib, icotinib, osimertinib, neratinib, gefitinib, cetuximab, panitumumab, lapatinib, dacomitinib, necitumumab, and vandetanib.

7. The method of claim 6, wherein the EGRF inhibitor is erlotinib.

8. The method of claim 7, wherein the erlotinib is present in culture media at a concentration between about 2 μM and 25 μM, inclusive, preferably between 5 μM and 10 μM, inclusive.

9. The method of claim 1 further comprising quantitating the level of CD55 expressed in the cultured hMSC.

10. A human mesenchymal stem cell (hMSC) prepared according to the method of claim 1.

11. A method of identifying a human mesenchymal stem cell (hMSC) for allogenic transplantation, the method comprising measuring the level of CD55 mRNA and/or the level of expression of CD55 on the surface of the hMSC, wherein the hMSC is identified as one suitable for transplantation if the level of CD55 mRNA and/or the level of expression of CD55 on the surface of the hMSC is higher than a minimum threshold of the level of CD55 mRNA and/or the level of expression of CD55 on the surface.

12. The method of claim 11, wherein the hMSC is isolated from a source selected from the group consisting of bone marrow, adipose tissue, amniotic fluid, endometrium, dental tissues, umbilical cord, Wharton's jelly, amniotic membrane, limb bud, menstrual blood, peripheral blood, placenta and fetal membrane, salivary gland, skin, foreskin, sub-amniotic umbilical cord lining membrane, and synovial fluid.

13. The method of claim 12, wherein the hMSC is isolated from adipose tissue.

14. The method of claim 11 further comprising administering the identified hMSC to a subject in need thereof.

15. The method of claim 14, wherein the subject is undergoing allogenic transplantation or is suffering from one or more symptoms associated with acute graft versus host disease.

16. The method of claim 11, wherein the minimum threshold is about 10%, 20%, 30%, 40%, 50%, 60%, 70%, 80%, or 90% of the level of CD55 mRNA and/or the level of expression of CD55 on BM-hMSCs.

17. A method comprising administering to a subject in need thereof an effective amount of human mesenchymal stem cells (hMSCs), having increased expression of CD55 mRNA and/or increased expression of CD55 on the surface of the hMSCs as compared to a minimum threshold of the level of CD55 mRNA and/or the level of expression of CD55,
wherein the subject is suffering from or at risk of developing one or more degenerative and/or immune disorders.

18. The method of claim 17, wherein the minimum threshold is at least 10%, 20%, 30%, 40%, 50%, 60%, 70%, 80%, or 90% of the level of CD55 mRNA and/or the level of expression of CD55 on BM-hMSCs.

19. The method of claim 17, wherein the hMSCs are isolated from adipose tissue.

20. The method of claim 17, wherein, prior to administering, the hMSCs are cultured with one or more active agents that induce or stimulate expression of CD55 mRNA and/or increase expression of CD55 on the surface of the hMSCs as compared to a control cell without one or more active agents.

21. The method of claim 20, wherein the active agent is an EGRF inhibitor or an HMG-CoA reductase inhibitor.

22. The method of claim 21, wherein the HMG-CoA reductase inhibitor is selected from the group consisting of atorvastatin, fluvastatin, lovastatin, pravastatin, rosuvastatin, simvastatin, and pitavastatin.

23. The method of claim 22, wherein the HMG-CoA reductase inhibitor is atorvastatin.

24. The method of claim 21, wherein the EGRF inhibitor is selected from the group consisting of erlotinib, afatinib, brigatinib, icotinib, osimertinib, neratinib, gefitinib, cetuximab, panitumumab, lapatinib, dacomitinib, necitumumab, and vandetanib.

25. The method of claim 24, wherein the EGRF inhibitor is erlotinib.

26. The method of claim 17, wherein the one or more degenerative and/or immune disorders are one or more neurodegenerative diseases and/or autoimmune diseases.

27. The method of claim 26, wherein the one or more neurodegenerative diseases are selected from the group consisting of amyotrophic lateral sclerosis, Parkinson's disease, muscular dystrophy, and Alzheimer disease.

28. The method of claim 26, wherein the one or more autoimmune diseases are selected from the group consisting of rheumatoid arthritis, Crohn's disease, ulcerative colitis, and Type 1 diabetes.

29. The method of claim 17, wherein the degenerative and immune disorder is graft versus host disease (GvHD).

30. The method of claim 29, wherein the degenerative and immune disorder is acute GvHD.
